(19) United States
(12) Patent Application Publication
(10) Pub. No.: US 2026/0209226 A1
(43) Pub. Date: Jul. 23, 2026

(54) HETEROCYCLIC PROPYL AMINE MODULATORS OF MONOAMINERGIC TRANSPORTERS

(71) Applicant: EmpathBio, Inc., New York, NY (US)

(72) Inventors: Alan C. GIBBS, Wyndmoor, PA (US); Robert B. PERNI, Marlborough, MA (US); Tanweer A. KHAN, Berlin (DE); Glenn SHORT, New York, NY (US)

(21) Appl. No.: 19/145,450

(22) PCT Filed: Jan. 5, 2024
(86) PCT No.: PCT/US2024/010486

§ 371 Date: Jul. 2, 2025

Related U.S. Application Data
(60) Provisional application No. 63/437,289, filed on Jan. 5, 2023.
(60) Provisional application No. 63/437,777, filed on Jan. 9, 2023.

(51) Int. Cl. C07D 471/04; A61K 31/423; A61K 31/428; A61K 31/437; C07D 261/20; C07D 263/56; C07D 277/62

(52) U.S. Cl. CPC C07D 471/04; A61K 31/423; A61K 31/428; A61K 31/437; C07D 261/20; C07D 263/56; C07D 277/62

(57) ABSTRACT

Provided herein are compounds of Formula (I): or a pharmaceutically acceptable salt thereof, wherein R₁, R₂, R₃, R₄, R₅, V W, X, Y, and Z are defined herein. Also provided are pharmaceutical compositions comprising a compound of Formula (I) or a pharmaceutically acceptable salt thereof, and methods of using a compound of Formula (I) or a pharmaceutically acceptable salt thereof, e.g., in the treatment of a mental heal disease or disorder.

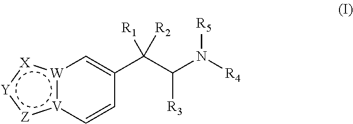

### CROSS REFERENCE TO RELATED APPLICATIONS

This application is related to and claims the benefit of priority to U.S. Provisional application Ser. No. 63/437,289, filed Jan. 5, 2023, and U.S. Provisional application Ser. No. 63/437,777, filed Jan. 9, 2023, both of which are Incorporated herein by reference in their entirety.

### FIELD

The disclosure relates to compounds, compositions, medicaments, methods and uses of heterocyclic propyl amine compounds that find use in the treatment, prevention, and/or management of one or more neurological disease or disorder, including various mental health diseases or disorders, and associated clinical symptoms.

### BACKGROUND

3,4-methylenedioxymethamphetamine (MDMA), also known as Ecstasy, is considered the prototype of a class of compounds called enactogens. Structurally, MDMA is a ring-substituted phenethylamine with a chiral center that gives rise to two enantiomeric stereoisomers: S(+)-MDMA and R(−)-MDMA. As typically described in the literature, the effects associated with the S-enantiomer resemble those of psychostimulants and are primarily mediated by dopaminergic and noradrenergic pathways, including increases in motor activity and euphoria, whereas effects associated with the R-enantiomer induces qualitative effects similar to classical psychedelics, such as ego-dissolution and perceptive alterations, mediated by serotonergic pathways, including direct 5-HT2A receptor agonism (Murnane et al., 2009).

Harnessing the biological activity of MDMA in an effective therapeutic treatment has been somewhat limited. This is due at least in part to a problematic pharmacokinetic profile related to non-proportional dose-dependency upon administration in humans. Studies have demonstrated that MDMA is metabolized to three main metabolites identified as MDA, HMMA, and HMA, which are found in the plasma in different proportions, depending on the concentration of the drug administered. At doses around 50-100 mg, HMMA predominates, while at doses between 125-150 mg MDMA predominates, indicating a possible saturation of its own metabolic pathways. The main hepatic enzymes involved in MDMA metabolism, as identified from in vitro experiments in human liver microsomes, are CYP1 A2, CYP2D6, and CYP3A4. Furthermore, stereochemistry appears to play a role in the non-renal pharmacokinetics of MDMA, with the S-enantiomer having a shorter half-life, lower peak plasma concentrations, and increased clearance (Green et al., 2003).

Accordingly, novel compounds and approaches that improve the pharmacokinetics of MDMA and MDA, and derivatives thereof, are needed in order to take advantage of the wide ranging pharmacologic properties of these compounds in treating various diseases and conditions.

### SUMMARY

In an aspect, the disclosure provides a compound of Formula (I):

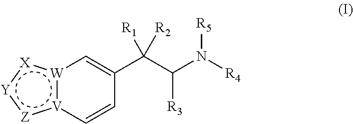

or a pharmaceutically acceptable salt thereof, wherein
V and W independently are C or N;
X, Y, and Z independently are O, S, CR₆, N, or NR₇;
wherein R₆ is hydrogen, deuterium, halogen, alkyl, fluoroalkyl, O-alkyl, O-fluoroalkyl, aryl, or benzyl;
   wherein R₇ is hydrogen, deuterium, alkyl, or fluoroalkyl;
R₁ and R₂ independently are hydrogen, deuterium, halogen, alkyl, fluoroalkyl, O-alkyl, or O-fluoroalkyl; or
R₁ and R₂ together are carbonyl; or
R₁ and R₂ together with the carbon atom to which they are attached form a cycloalkyl;
R₃ is hydrogen, deuterium, halogen, alkyl, fluoroalkyl, O-alkyl, or O-fluoroalkyl; and
   R₄ and R₅ independently are hydrogen, deuterium, alkyl, or fluoroalkyl; or one of R₄ or R₅ is

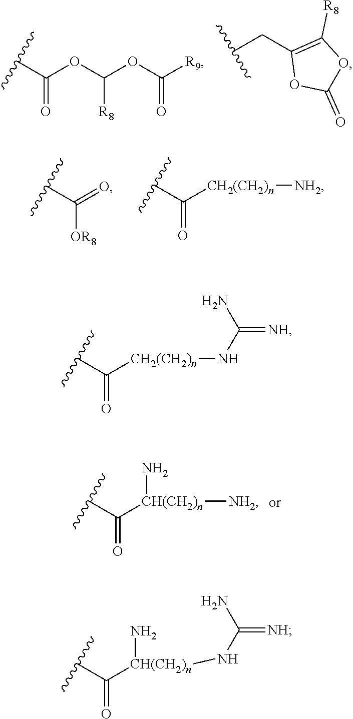

wherein R₈ and R₉ independently are alkyl; and
wherein n is an integer from 2-5.

In some embodiments relating to compounds of Formula (I), X, Y, and Z independently are CR₆ or NR₇; wherein R₆ is C₁-C₆ alkyl, C₁-C₆ fluoroalkyl, O—(C₁-C₆ alkyl), O—(C₁-C₆ fluoroalkyl); wherein R₇ is C₁-C₆ alkyl or C₁-C₆ fluoroalkyl; R₁ and R₂ independently are C₁-C₆ alkyl, C₁-C₆ fluoroalkyl, O—(C₁-C₆ alkyl), O—(C₁-C₆ fluoroalkyl); or R₁ and R₂ together with the carbon atom to which they are attached form a C₃-C₇ cycloalkyl; R₃ is C₁-C₆ alkyl, C₁-C₆ fluoroalkyl, O—(C₁-C₆ alkyl), O—(C₁-C₆ fluoroalkyl); and R₄ and R₅ independently are C₁-C₆ alkyl or C₁-C₆ fluoroalkyl; or one of R₄ or R₅ is:

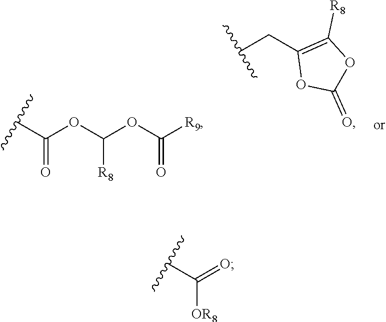

wherein R₈ and R₉ independently are C₁-C₆ alkyl.

In some embodiments relating to compounds of Formula (I), R₃ is C₁-C₆ alkyl. In some further embodiments, R₃ is methyl.

In some embodiments relating to compounds of Formula (I), R₄ or R₅ is C₁-C₆ alkyl. In some further embodiments, R₄ or R₅ is methyl.

In some embodiments relating to compounds of Formula (I), R₄ and R₅ are hydrogen.

In some embodiments relating to compounds of Formula (I), at least two of X, W, X, Y, and Z are selected from N and S. In some further embodiments, at least three of X, W, X, Y, and Z are selected from N and S. In some further embodiments, V and X are N. In some further embodiments, X is S and Z is N. In some further embodiments, V, X, and Z are N. In some further embodiments, X is N and Z is S. In some further embodiments, V is C. In some further embodiments, V is N. In some further embodiments, W is C. In some further embodiments, X is N. In some further embodiments, X is S. In some further embodiments, Y is CH. In some further embodiments, Z is CH. In some further embodiments, Z is N. In some further embodiments, Z is S.

In some embodiments relating to compounds of Formula (I), R₁ and R₂ are hydrogen.

In another aspect the disclosure provides a pharmaceutical composition, comprising any of the compounds of the above-described aspects and embodiments, and a pharmaceutically acceptable carrier.

In another aspect the disclosure provides a method for treating a neurological disorder, comprising administering to a subject in need thereof an effective amount of the compound or pharmaceutical composition of any of the above described aspects and embodiments. In further embodiments, the neurological disorder is post-traumatic stress disorder. In further embodiments, the neurological disorder is an eating disorder including anorexia nervosa or bulimia nervosa. In further embodiments, the neurological disorder is obsessive-compulsive disorder (OCD).

Additional aspects and embodiments in accordance with the disclosure will be apparent to one of skill in the art in light of the following description.

### DETAILED DESCRIPTION

Before the disclosed various aspects and embodiments relating to compounds, compositions, formulations, methods and materials that fall within the scope of the disclosure are described, it is to be understood that the specific description detailed herein is not limited to those specific embodiments, and can vary as apparent to one of skill in the art. It is also to be understood that the terminology used herein is generally provided for the purpose of describing certain particular aspects and embodiments only and, unless specifically defined herein, is not intended to be limiting.

### Definitions

Throughout this specification, unless the context requires otherwise, the word “comprise” and “include” and variations (e.g., “comprises,” “comprising,” “includes,” “including”) will be understood to imply the inclusion of a stated component, feature, element, or step or group of components, features, elements or steps but not the exclusion of any other integer or step or group of integers or steps.

As used in the specification and the appended claims, the singular forms “a,” “an” and “the” include plural referents unless the context clearly dictates otherwise.

The following terms and expressions used herein have the indicated meanings.

Terms used herein may be preceded and/or followed by a single dash, “—”, or a double dash, “═”, to indicate the bond order of the bond between the named substituent and its parent moiety; a single dash indicates a single bond and a double dash indicates a double bond. In the absence of a single or double dash it is understood that a single bond is formed between the substituent and its parent moiety.

An “alkyl” group refers to a fully saturated straight or branched chain hydrocarbon containing from 1 to 12 carbon atoms, which is attached to a molecule by a single bond. Alkyl groups can include C₁-C₁₂ alkyl, C₁-C₁₀ alkyl, C₁-C₆ alkyl, C₁₋₅ alkyl all of which are inclusive of C₄ alkyls, C₃ alkyls, C₂ alkyls and C₁ alkyl(methyl). Non-limiting examples of alkyl include methyl, ethyl, n-propyl, iso-propyl, n-butyl, sec-butyl, iso-butyl, tert-butyl, n-pentyl, isopentyl, neopentyl, t-amyl, n-hexyl, 3-methylhexyl, 2,2-dimethylpentyl, 2,3-dimethylpentyl, n-heptyl, n-octyl, n-nonyl, n-decyl, n-undecyl, and n-dodecyl. In accordance with some example embodiments an alkyl group can be optionally substituted.

“Alkylene” refers to a saturated, straight or branched bivalent alkyl group. An “alkylene chain” refers to a polymethylene group, i.e., —(CH₂)ₙ—, wherein n is a positive integer which, in certain embodiments, can be from one to six, from one to four, from one to three, from one to two, or from two to three. A substituted alkylene chain is a polymethylene group in which one or more methylene hydrogen atoms is replaced with a substituent. Suitable substituents include those described below for a substituted aliphatic group. An alkylene chain also may be substituted at one or more positions with an aliphatic group or a substituted aliphatic group.

The term “alkenyl” refers to a straight or branched chain hydrocarbon containing from 2 to 12 carbons and containing at least one carbon-carbon double bond. Alkenyl groups can include C₂-C₁₂ alkenyl, C₂-C₁₀ alkenyl, C₂-C₆ alkenyl, C₂₋₅ alkenyl all of which are inclusive of C₄ alkenyls, C₃ alkenyls, and C₂ alkenyls. Non-limiting examples of alkenyl include ethenyl (vinyl), 1-propenyl, 2-propenyl (allyl), isopropenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. In accordance with some example embodiments an alkenyl group can be optionally substituted.

The term “alkynyl” refers to a straight or branched chain hydrocarbon group containing from 2 to 12 carbon atoms and containing at least one carbon-carbon triple bond. Alkynyl groups can include C₂-C₁₂ alkynyl, C₂-C₁₀ alkynyl, C₂-C₆ alkynyl, C₂-C₅ alkynyl all of which are inclusive of C₄ alkynyl, C₃ alkynyl, and C₂ alkynyl. Non-limiting examples of alkynyl include, but are not limited, to acetylenyl (ethynyl), propynyl (i.e., 1-propynyl, 2-propynyl), butynyl, pentynyl, and the like. In accordance with some example embodiments an alkynyl group can be optionally substituted.

“Alkoxy” refers to a group of the formula —OR, where R is an alkyl, alkenyl, or alkynyl group, as defined herein, appended to the parent molecular moiety through the oxygen atom. Non-limiting examples of alkoxy groups include methoxy, ethoxy, propoxy, 2-propoxy, butoxy, tert-butoxy, pentyloxy, and hexyloxy. In accordance with some example embodiments an alkoxy group can be optionally substituted.

The term “aryl” refers to a stable monocyclic (i.e., phenyl), bicyclic, tricyclic or tetracyclic ring system containing 6 to 18 carbon atoms and at least one aromatic ring in the ring system. An aryl group can include fused and/or bridged ring systems. Non-limiting examples of aryl include aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. In accordance with some example embodiments an aryl group can be optionally substituted.

The term “cycloalkyl” refers to a stable monocyclic, bicyclic, polycyclic, or spirocyclic fully saturated ring system typically comprising from 3 to 20 carbon atoms. Monocyclic ring systems are cyclic hydrocarbon groups that in some embodiments contain from 3 to 10 carbon atoms. Non-limiting examples of monocyclic cycloalkyls include cyclopropyl, cyclobutyl, cyclopentyl, cyclopentenyl, cyclohexyl, cycloheptyl, and cyclooctyl. Bicyclic cycloalkyl ring systems are bridged monocyclic rings or fused bicyclic rings. Bridged monocyclic rings contain a monocyclic cycloalkyl ring where two non-adjacent carbon atoms of the monocyclic ring are linked by an alkylene bridge of between one and three additional carbon atoms (i.e., a bridging group of the form —(CH₂)_{w}, where w is 1, 2, or 3). Non-limiting examples of bicyclic and polycyclic ring systems include, but are not limited to, bicyclo[3.1.1]heptane, bicyclo[2.2.1]heptane, bicyclo[2.2.2]octane, bicyclo[3.2.2]nonane, bicyclo[3.3.1]nonane, and bicyclo[4.2.1]nonane, adamantyl, norbornyl, decalinyl, 7,7-dimethyl-bicyclo[2.2.1]heptanyl, and the like. In accordance with some example embodiments a cycloalkyl group can be optionally substituted.

“Cycloalkenyl” refers to a stable non-aromatic monocyclic, bicyclic, or polycyclic hydrocarbon consisting solely of carbon and hydrogen atoms, having one or more carbon-carbon double bonds, which can include fused or bridged ring systems, having from 3 to 20 carbon atoms, preferably having from 3 to 10 carbon atoms. Monocyclic cycloalkenyls include, for example, cyclopentenyl, cyclohexenyl, cycloheptenyl, cycloctenyl, and the like. Unless otherwise stated specifically in the specification, a cycloalkenyl group can be optionally substituted.

The term “halo” or “halogen” refers to one or a combination of —Cl, —Br, —I, or —F.

The terms “haloalkyl,” “haloalkenyl,” “haloalkynyl,” and “haloalkoxy” refer to an alkyl, alkenyl, alkynyl, or alkoxy group, as defined above, which is substituted with one or more halogen atoms at any available position. In accordance with some example embodiments any of these “halo-” groups can be optionally substituted.

The terms “heterocyclyl” and “heterocycle” refer to a 3- to 20-membered monocyclic, bicyclic, polycyclic, or spirocycllc ring system that may be saturated, unsaturated, or aromatic and that includes from 1 to 6 heteroatoms, N, O, or S. Monocyclic heterocycles include 3, 4, 5, 6, and 7 membered-rings containing at least 1 heteroatom independently selected from the group consisting of 0, N, and S. The heterocycle is connected to the parent molecular moiety through any carbon atom or any nitrogen atom contained within the heterocycle. Non-limiting examples of monocyclic heterocycles include azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. Non-limiting examples of bicyclic heterocycles include 2,3-dihydrobenzofuran-2-yl, 2,3-dihydrobenzofuran-3-yl, indolin-1-yl, indolln-2-yl, indolin-3-yl, 2,3-dihydrobenzothien-2-yl, decahydroquinolinyl, decahydroisoquinolinyl, octahydro-1H-indolyl, and octahyd robe nzofu ranyl.

The term “oxo” as used herein means a ═O group.

The term “thia” as used herein means a ═S group.

The term “saturated” as used herein means the referenced chemical structure does not contain any multiple carbon-carbon bonds. For example, a saturated cycloalkyl group as defined herein includes cyclohexyl, cyclopropyl, and the like.

The term “unsaturated” as used herein means the referenced chemical structure contains at least one multiple carbon-carbon bond, but is not aromatic. For example, a unsaturated cycloalkyl group as defined herein includes cyclohexenyl, cyclopentenyl, cyclohexadienyl, and the like.

The term “substituted”, as used herein, means that a hydrogen radical of the designated moiety is replaced with the radical of a specified substituent, provided that the substitution results in a stable or chemically feasible compound. The term “substitutable”, when used in reference to a designated atom, means that attached to the atom is a hydrogen radical, which can be replaced with the radical of a suitable substituent.

As used herein, “treat” or “treating” means accomplishing one or more of the following: (a) reducing the severity of the disorder; (b) limiting or preventing development of symptoms characteristic of the disorder(s) being treated; (c) inhibiting worsening of symptoms characteristic of the disorder(s) being treated; (d) limiting or preventing recurrence of the disorder(s) in patients that have previously had the disorder(s); and (e) limiting or preventing recurrence of symptoms in patients that were previously symptomatic for the disorder(s).

The phrase “one or more” substituents, as used herein, refers to a number of substituents that equals from one to the maximum number of substituents possible based on the number of available bonding sites, provided that the above conditions of stability and chemical feasibility are met. Unless otherwise indicated, an optionally substituted group may have a substituent at each substitutable position of the group, and the substituents may be either the same or different. As used herein, the term “independently selected” means that the same or different values may be selected for multiple instances of a given variable in a single compound.

When a range of values is listed, it is intended to encompass each value and sub-range within the range. For example, “C₁-C₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

It will be apparent to one skilled in the art that certain compounds of this disclosure may exist in tautomeric forms, all such tautomeric forms of the compounds being within the scope of the disclosure. Unless otherwise stated, structures depicted herein are also meant to include all stereochemical forms of the structure; i.e., the Rand S configurations for each asymmetric center. Therefore, single stereochemical isomers as well as enantiomeric and diastereomeric mixtures of the present compounds are within the scope of the disclosure. Both the Rand the S stereochemical isomers, as well as all mixtures thereof, are included within the scope of the disclosure.

In view of the present disclosure, the methods and compositions described herein can be configured by the person skilled in the art to meet the desired need. In general, the disclosed materials and methods provide improvements in treatment of mental health disorders.

### Compounds

In some embodiments, the disclosure provides a compound of Formula (I):

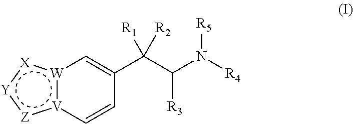

or a pharmaceutically acceptable salt thereof,
wherein,
V and W independently are C or N;
X, Y, and Z independently are O, S, CR₆, N, or NR₇;
   wherein R₆ is hydrogen, deuterium, halogen, alkyl, fluoroalkyl, O-alkyl, O-fluoroalkyl, aryl, or benzyl;
   wherein R₇ is hydrogen, deuterium, alkyl, or fluoroalkyl;
R₁ and R₂ independently are hydrogen, deuterium, halogen, alkyl, fluoroalkyl, O-alkyl, or O-fluoroalkyl; or
R₁ and R₂ together are carbonyl; or
R₁ and R₂ together with the carbon atom to which they are attached form a cycloalkyl;
R₃ is hydrogen, deuterium, halogen, alkyl, fluoroalkyl, O-alkyl, or O-fluoroalkyl; and
R₄ and R₅ independently are hydrogen, deuterium, alkyl, or fluoroalkyl; or one of R₄ or R₅ is

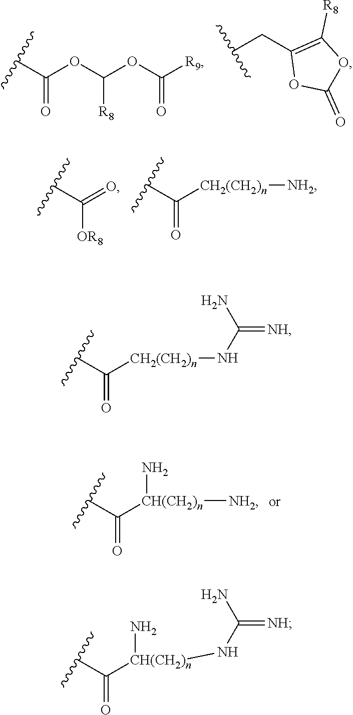

wherein R₈ and R₉ independently are alkyl; and
wherein n is an integer from 2-5.

In some embodiments, the compound of Formula (I) is:

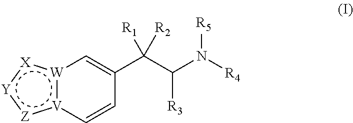

X, Y, and Z independently are CR₆ or NR₇;
   wherein R₆ is C₁-C₆ alkyl, C₁-C₆ fluoroalkyl, O—(C₁-C₆ alkyl), or O—(C₁-C₆ fluoroalkyl);
wherein R₇ is C₁-C₆ alkyl or C₁-C₆ fluoroalkyl;
R₁ and R₂ independently are C₁-C₆ alkyl, C₁-C₆ fluoroalkyl, O—(C₁-C₆ alkyl), or O—(C₁-C₆ fluoroalkyl); or
R₁ and R₂ together with the carbon atom to which they are attached form a C₃-C₇ cycloalkyl;
R₃ is C₁-C₆ alkyl, C₁-C₆ fluoroalkyl, O—(C₁-C₆ alkyl), or O—(C₁-C₆ fluoroalkyl); and
R₄ and R₅ independently are C₁-C₆ alkyl or C₁-C₆ fluoroalkyl; or
one of R₄ or R₅ is

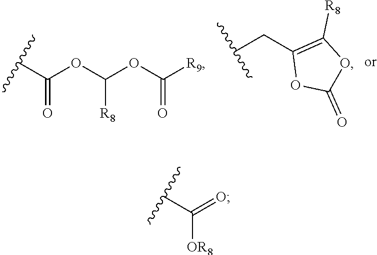

wherein R₈ and R₉ independently are C₁-C₆ alkyl.

In some embodiments, the compound of Formula (I) is a compound of Formula (I-A):

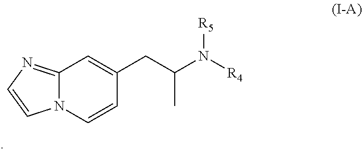

or a pharmaceutically acceptable salt thereof, wherein,
R₄ and R₅ independently are hydrogen, deuterium, C₁-C₆ alkyl or C₁-C₆ fluoroalkyl; or
one of R₄ or R₅ is

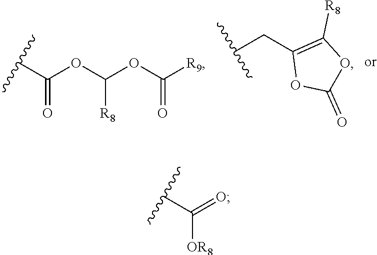

wherein R₈ and R₉ independently are C₁-C₆ alkyl.

In some embodiments, the compound of Formula (I) is a compound of Formula (I-B):

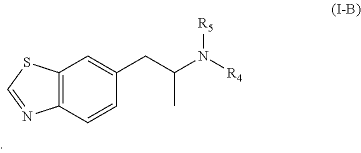

or a pharmaceutically acceptable salt thereof, wherein,
R₄ and R₅ independently are hydrogen, deuterium, C₁-C₆ alkyl or C₁-C₆ fluoroalkyl; or
one of R₄ or R₅ is

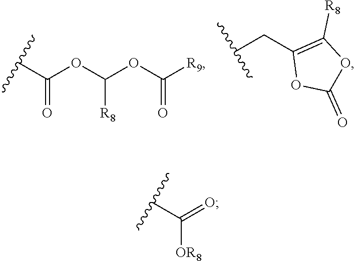

wherein R₈ and R₉ independently are C₁-C₆ alkyl.

In some embodiments, the compound of Formula (I) is a compound of Formula (I C):

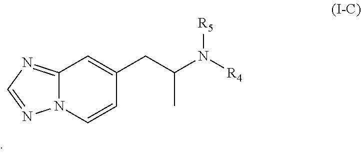

or a pharmaceutically acceptable salt thereof, wherein,
R₄ and R₅ independently are hydrogen, deuterium, C₁-C₆ alkyl or C₁-C₆ fluoroalkyl; or
one of R₄ or R₅ is

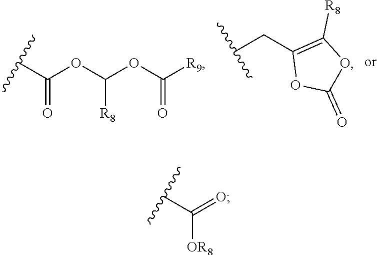

wherein R₈ and R₉ independently are C₁-C₆ alkyl.

In some embodiments, the compound of Formula (I) is a compound of Formula (I-D):

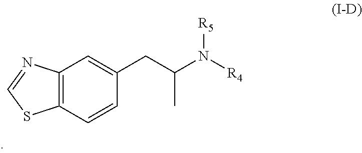

or a pharmaceutically acceptable salt thereof, wherein,
R₄ and R₅ independently are hydrogen, deuterium, C₁-C₆ alkyl or C₁-C₆ fluoroalkyl; or
one of R₄ or R₅ is

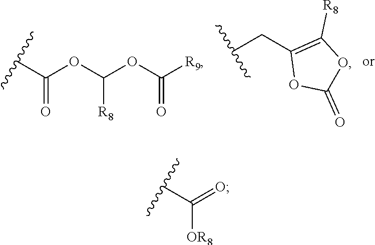

wherein R₈ and R₉ independently are C₁-C₆ alkyl.

In some embodiments of the compounds of Formula (I), (I-A), (I-B), (I-C), or (I-D), R₁ and R₂ independently are hydrogen, deuterium, C₁-C₆ alkyl, C₁-C₆ fluoroalkyl, O—(C₁-C₆ alkyl), or O—(C₁-C₆ fluoroalkyl).

In some embodiments of the compounds of Formula (I), (I-A), (I-B), (I-C), or (I-D), R₁ and R₂ together are carbonyl.

In some embodiments of the compounds of Formula (I), (I-A), (I-B), (I-C), or (I-D), R₁ and R₂ together with the carbon atom to which they are attached form a C₃-C₇ cycloalkyl.

In some embodiments of the compounds of Formula (I), (I-A), (I-B), (I-C), or (I-D), R₁ and R₂ are hydrogen.

In some embodiments of the compounds of Formula (I), (I-A), (I-B), (I-C), or (I-D), R₃ is hydrogen, deuterium, halogen, C₁-C₆ alkyl, C₁-C₆ fluoroalkyl, O—(C₁-C₆ alkyl), or O—(C₁-C₆ fluoroalkyl).

In some embodiments of the compounds of Formula (I), (I-A), (I-B), (I-C), or (I-D), R₃ is C₁-C₆ alkyl.

In some embodiments of the compounds of Formula (I), (I-A), (I-B), (I-C), or (I-D), R₃ is methyl.

In some embodiments of the compounds of Formula (I), (I-A), (I-B), (I-C), or (I-D), R₄ and R₅ independently are hydrogen, deuterium, C₁-C₆ alkyl or C₁-C₆ fluoroalkyl.

In some embodiments of the compounds of Formula (I), (I-A), (I-B), (I-C), or (I-D), R₄ or R₅ is C₁-C₆ alkyl.

In some embodiments of the compounds of Formula (I), (I-A), (I-B), (I-C), or (I-D), R₄ or R₅ is methyl.

In some embodiments of the compounds of Formula (I), (I-A), (I-B), (I-C), or (I-D), R₄ and R₅ are hydrogen.

In some embodiments of the compounds of Formula (I), (I-A), (I-B), (I-C), or (I-D), one of R₄ or R₅ is

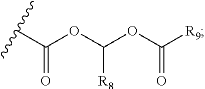

wherein R₈ and R₉ independently are C-C₆ alkyl.

In some embodiments of the compounds of Formula (I), (I-A), (I-B), (I-C), or (I-D), one of R₄ or R₅ is

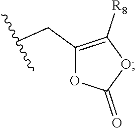

wherein R₈ is C₁-C₆ alkyl.

In some embodiments of the compounds of Formula (I), (I-A), (I-B), (I-C), or (I-D), one of R₄ or R₅ is

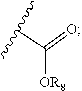

wherein R₈ is C₁-C₆ alkyl.

In some embodiments of the compounds of Formula (I), (I-A), (I-B), (I-C), or (I-D), one of R₄ or R₅ is

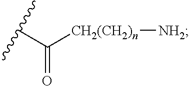

wherein n is an integer from 2-5.

In some embodiments of the compounds of Formula (I), (I-A), (I-B), (I-C), or (I-D), one of R₄ or R₅ is

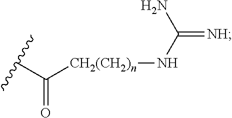

wherein n is an integer from 2-5.

In some embodiments of the compounds of Formula (I), (I-A), (I-B), (I-C), or (I-D), one of R₄ or R₅ is

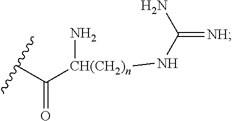

wherein n is an integer from 2-5.

In some embodiments of the compounds of Formula (I), (I-A), (I-B), (I-C), or (I-D), one of R₄ or R₅ is; wherein n is an integer from 2-5.

In some embodiments of the compounds of Formula (I), (I-A), (I-B), (I-C), or (I-D), V and W independently are C or N.

In some embodiments of the compounds of Formula (I), (I-A), (I-B), (I-C), or (I-D), X, Y, and Z independently are O, S, CR₆, N, or NR₇; wherein R₆ is C₁-C₆ alkyl, C₁-C₆ fluoroalkyl, O—(C₁-C₆ alkyl), O—(C₁-C₆ fluoroalkyl), and R₇ is C₁-C₆ alkyl or C₁-C₆ fluoroalkyl.

In some embodiments of the compounds of Formula (I), (I-A), (I-B), (I-C), or (I-D), at least two of V, W, X, Y, and Z are selected from N and S.

In some embodiments of the compounds of Formula (I), (I-A), (I-B), (I-C), or (I-D), at least three of V, W, X, Y, and Z are selected from N and S.

In some embodiments of the compounds of Formula (I), (I-A), (I-B), (I-C), or (I-D), V and X are N.

In some embodiments of the compounds of Formula (I), (I-A), (I-B), (I-C), or (I-D), X is S and Z is N.

In some embodiments of the compounds of Formula (I), (I-A), (I-B), (I-C), or (I-D), V, X, and Z are N.

In some embodiments of the compounds of Formula (I), (I-A), (I-B), (I-C), or (I-D), X is N and Z is S.

In some embodiments of the compounds of Formula (I), (I-A), (I-B), (I-C), or (I-D), V is C.

In some embodiments of the compounds of Formula (I), (I-A), (I-B), (I-C), or (I-D), V is N.

In some embodiments of the compounds of Formula (I), (I-A), (I-B), (I-C), or (I-D), W is C.

In some embodiments of the compounds of Formula (I), (I-A), (I-B), (I-C), or (I-D), X is N.

In some embodiments of the compounds of Formula (I), (I-A), (I-B), (I-C), or (I-D), X is S.

In some embodiments of the compounds of Formula (I), (I-A), (I-B), (I-C), or (I-D), Y is CH.

In some embodiments of the compounds of Formula (I), (I-A), (I-B), (I-C), or (I-D), Z is CH.

In some embodiments of the compounds of Formula (I), (I-A), (I-B), (I-C), or (I-D), Z is N.

In some embodiments of the compounds of Formula (I), (I-A), (I-B), (I-C), or (I-D), Z is S.

In certain non-limiting example embodiments, the compounds are selected from the following group in Table 1.

TABLE 1
Non-limiting Exemplary Compounds

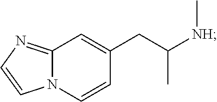

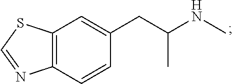

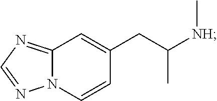

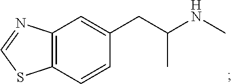

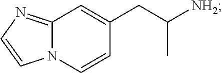

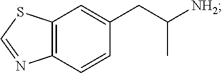

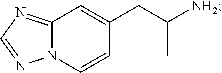

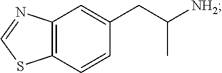

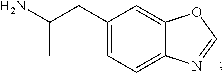

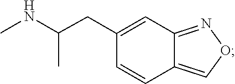

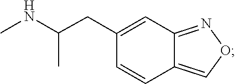

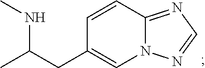

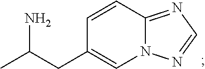

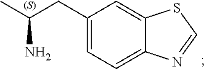

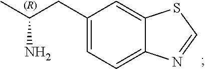

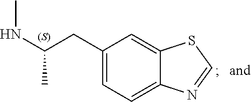

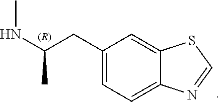

In some embodiments of the compounds of Formula (I), (I-A), (I-B), (I-C), or (I-D), the compound is not (i.e., specifically exclude):

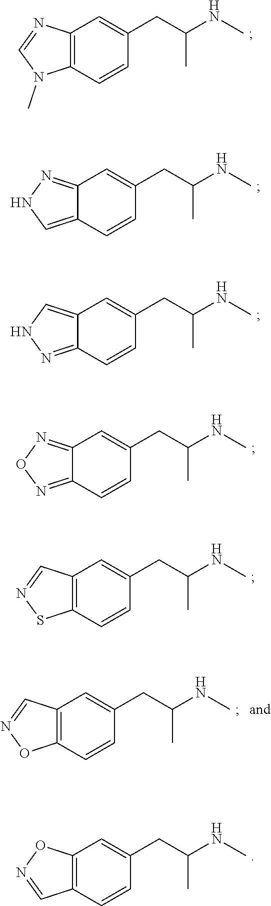

### Salts

The compounds in accordance with the disclosure include pharmaceutically acceptable salts thereof, suitable for use in contact with the tissues of patients without undue toxicity, irritation, allergic response, and the like, commensurate with a reasonable benefit/risk ratio, and effective for their intended use, of the compounds of the invention. The term “salts” refers to the relatively non-toxic, inorganic and organic acid addition salts of compounds of the present invention. These salts can be prepared in situ during the final isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate, mesylate, glucoheptonate, lactobionate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See, for example, Berge S. M. et al., “Pharmaceutical Salts,” J. Pharm. Sci., 1977; 66:1-19 which is incorporated herein by reference.). In such embodiments, and as described below, the disclosure relates to the preparation of a medicament comprising one or more of the compounds disclosed herein, in combination with a pharmaceutically acceptable salt, carriers, diluents, and/or adjuvants.

### Methods, Uses, and Therapeutics Applications

In some aspects and embodiments, the present disclosure provides a method of treating or preventing neurological disorders in a subject in need thereof, the method comprising administering to the subject a therapeutically effective amount of a compound disclosed herein, e.g., a compound of Formula (I), (I-A), (I-B), (I-C), (I-D), or Table 1, or a pharmaceutically acceptable salt thereof. In such embodiments, the disclosure relates to uses of one or more of the compounds disclosed herein, for the treatment or prevention of one or more neurological disorders as described herein.

In some embodiments, the neurological disorder is a mood disorder. In embodiments, the mood disorder is clinical depression, postnatal depression or postpartum depression, perinatal depression, atypical depression, melancholic depression, psychotic major depression, cationic depression, seasonal affective disorder, dysthymia, double depression, depressive personality disorder, borderline personality disorder, Cluster C personality disorders (e.g., avoidant personality disorder, dependent personality disorder, and obsessive-compulsive personality disorder), recurrent brief depression, major depressive disorder, minor depressive disorder, bipolar disorder or manic depressive disorder, depression caused by chronic medical conditions, treatment-resistant depression, refractory depression, suicidality, suicidal ideation, or suicidal behavior. In embodiments, the method described herein provides therapeutic effect to a subject suffering from depression (e.g., moderate or severe depression). In some embodiments, the mood disorder is associated with neuroendocrine diseases and disorders, neurodegenerative diseases and disorders (e.g., epilepsy), movement disorders, tremor (e.g., Parkinson's Disease), or women's health disorders or conditions. In embodiments the mood disorder is depression. In embodiments, the mood disorder is treatment-resistant depression or major depressive disorder. In some embodiments, the mood disorder is major depressive disorder. In embodiments, the mood disorder is treatment-resistant depression.

In some embodiments, the present disclosure provides a use and/or method of treating or preventing post-traumatic stress disorder (PTSD), mood disorders, one or more personality disorders (e.g., borderline personality disorder, Cluster C personality disorders (e.g., avoidant personality disorder, dependent personality disorder, and obsessive-compulsive personality disorder), general anxiety disorder, addictive disorders, and/or drug dependence in a subject in need thereof, the method comprising administering to the subject a therapeutically effective amount of a compound disclosed herein, e.g., a compound of Formula (I), (I-A), (I-B), (I-C), (I-D), or Table 1, or a pharmaceutically acceptable salt there, or a pharmaceutical composition thereof.

In embodiments, the compounds of the present disclosure are used to treat PTSD. In embodiments, the compounds of the present disclosure are used for induction and maintenance therapy to treat PTSD. In embodiments, the compounds of the present disclosure are used to treat PTSD with an improved safety profile when compared to treatment with the entactogenic, oneirophrenic or psychedelic compound (e.g. MDMA or related compound, psilocybin or dimethyltryptamine) alone. In embodiments, the compounds of the present disclosure are used for induction and maintenance therapy to treat PTSD with an improved safety profile when compared to treatment with the entactogenic, oneirophrenic or psychedelic compound (e.g. MDMA or related compound, psilocybin or dimethyltryptamine) alone.

In embodiments, the compounds of the present disclosure are used to treat behavioral or mood disorders. Examples of behavioral or mood disorders include anxiety, such as social anxiety in autistic subjects (e.g. autistic adults) and anxiety related to life-threatening illnesses, stress (where moderation thereof is measured, for example, by effects on amygdala responses). In some embodiments, the anxiety disorder is panic disorder, obsessive-compulsive disorder, or general anxiety disorder. Other examples include lack of motivation, attention, accuracy, speed of response, perseveration, and/or cognitive engagement. Further examples include depression (e.g., MDD or TRD), attention disorders, disorders of executive function and/or cognitive engagement, obsessive compulsive disorder, bipolar disorder, panic disorder, phobia, schizophrenia, psychopathy, antisocial personality disorder and/or neurocognitive disorders.

In embodiments, the compounds the present disclosure are used to treat an addictive disorder. In embodiments, the addictive disorder is alcohol abuse, substance abuse, smoking, or obesity. In embodiments, the disorder is an eating disorder (anorexia nervosa, bulimia nervosa, binge eating disorder, etc.) or an auditory disorder.

In embodiments, the disorder is borderline personality disorder or a Cluster C personality disorder comprising one or more of avoidant personality disorder, dependent personality disorder, or obsessive-compulsive personality disorder.

In embodiments, the disorder is an impulsive disorder. In embodiments, the impulsive disorder is attention deficit hyperactivity disorder (ADHD), attention deficit disorder (ADD), Tourette's syndrome or autism.

In embodiments, the disorder is a compulsive disorder. In embodiments, the compulsive disorder is obsessive compulsive disorder (OCD), gambling, or aberrant sexual behavior.

In embodiments, the disorder is a personality disorder. In embodiments, the personality disorder is conduct disorder, antisocial personality, or aggressive behavior.

### Pharmaceutical Compositions

In some aspects and embodiments of the disclosure, a pharmaceutical composition comprising a therapeutically effective amount of one or more compounds of the present disclosure (e.g., a compound of Formula (I). (I-A), (I-B), (I-C), (I-D), or Table 1) or a pharmaceutically acceptable salt thereof and a pharmaceutically acceptable excipient is provided. In accordance with such aspects, the disclosure provides for the manufacture of a medicament comprising a therapeutically effective amount of one or more compounds of the present disclosure (e.g., a compound of Formula (I), (I-A), (I-B), (I-C), (I-D), or Table 1).

For administration as compositions, including pharmaceutical compositions, the compounds are ordinarily combined with one or more carriers, diluents, and/or adjuvants appropriate for the indicated route of administration. The compounds may be mixed with lactose, sucrose, starch powder, cellulose esters of alkanoic acids, stearic acid, talc, magnesium stearate, magnesium oxide, sodium and calcium salts of phosphoric and sulphuric acids, acacia, gelatin, sodium alginate, polyvinylpyrrolidine, and/or polyvinyl alcohol, and tableted or encapsulated for conventional administration. Alternatively, the compounds of this invention may be dissolved in saline, water, polyethylene glycol, propylene glycol, carboxymethyl cellulose colloidal solutions, ethanol, corn oil, peanut oil, cottonseed oil, sesame oil, tragacanth gum, and/or various buffers. Other adjuvants and modes of administration are well known in the pharmaceutical art. The carrier or diluent may include time delay material, such as glyceryl monostearate or glyceryl distearate alone or with a wax, or other materials well known in the art.

The compounds disclosed herein can be administered as the sole active pharmaceutical agent, or they can be used in combination with one or more other compounds useful for carrying out the methods and uses. When used or administered as a combination, the therapeutic agents can be formulated as separate compositions that are given at the same time or different times, or the therapeutic agents can be given as a single composition.

The compounds can be prepared in a solid form (including granules, powders or suppositories) or in a liquid form (e.g., solutions, suspensions, or emulsions). The disclosed compounds may be applied in a variety of solutions and may be subjected to conventional pharmaceutical operations such as sterilization and/or may contain conventional adjuvants, such as preservatives, stabilizers, wetting agents, emulsifiers, buffers, etc.

The disclosed compounds may be administered orally, topically, parenterally, by inhalation or spray or rectally in dosage unit formulations containing conventional non-toxic pharmaceutically acceptable carriers, adjuvants, and vehicles. The term parenteral as used herein includes percutaneous, subcutaneous, intravascular (e.g., intravenous), intramuscular, or intrathecal injection or infusion techniques and the like. One or more compounds in accordance with the disclosure may be present in association with one or more non-toxic pharmaceutically acceptable carriers and/or diluents and/or adjuvants, and if desired other active ingredients. Such pharmaceutical compositions may be in a form suitable for oral use, for example, as tablets, troches, lozenges, aqueous or oily suspensions, dispersible powders or granules, emulsion, hard or soft capsules, or syrups or elixirs.

Compositions intended for oral use may be prepared according to any method known to the art for the manufacture of pharmaceutical compositions and such compositions may contain one or more agents selected from the group consisting of sweetening agents, flavoring agents, coloring agents and preservative agents in order to provide palatable preparations. Tablets contain the active ingredient in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques. In some cases such coatings may be prepared by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in admixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents may be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl p-hydroxybenzoate, one or more coloring agents, one or more flavoring agents, and one or more sweetening agents, such as sucrose or saccharin.

Oily suspensions may be formulated by suspending the active ingredients in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents and flavoring agents may be added to provide palatable oral preparations. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, may also be present.

Pharmaceutical compositions in accordance with the disclosure may also be in the form of oil-in-water emulsions. The oily phase may be a vegetable oil or a mineral oil or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol, anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol, glucose or sucrose. Such formulations may also contain a demulcent, a preservative, and flavoring and coloring agents. The pharmaceutical compositions may be in the form of a sterile injectable aqueous or oleaginous suspension. This suspension may be formulated according to the known art using those suitable dispersing or wetting agents and suspending agents that have been mentioned above. The sterile injectable preparation may also be a sterile injectable solution or suspension in a non toxic parentally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds and pharmaceutical compositions in accordance with the disclosure may also be administered in the form of suppositories, e.g., for rectal administration of the drug. These compositions can be prepared by mixing the drug with a suitable non-irritating excipient that is solid at ordinary temperatures but liquid at the rectal temperature and will therefore melt in the rectum to release the drug. Such materials include cocoa butter and polyethylene glycols.

Compounds and pharmaceutical compositions in accordance with the disclosure may be administered parenterally in a sterile medium. The drug, depending on the vehicle and concentration used, can either be suspended or dissolved in the vehicle. Advantageously, adjuvants such as local anesthetics, preservatives and buffering agents can be dissolved in the vehicle.

The amount of compound(s) administered will depend upon a variety of factors, including, for example, the particular indication being treated, the mode of administration, whether the desired benefit is prophylactic or therapeutic, the severity of the indication being treated, the age and weight of the patient, the bioavailability of the particular compound(s), the metabolism rate and efficiency of the compound under the selected route of administration, etc. Determination of an effective dosage of compound(s) for a particular use and mode of administration is well within the capabilities of those skilled in the art. Effective dosages may be estimated initially from in vitro activity and metabolism assays. For example, an initial dosage of compound for use in animals may be formulated to achieve a circulating blood or serum concentration of the metabolite active compound that is at or above an IC₅₀ of the particular compound as measured in as in vitro assay. Calculating dosages to achieve such circulating blood or serum concentrations taking into account the bioavailability of the particular compound via the desired route of administration is well within the capabilities of skilled artisans. Initial dosages of compound can also be estimated from in vivo data, such as animal models. Animal models useful for testing the efficacy of the active metabolites to treat or prevent the various diseases described above are well-known in the art. Animal models suitable for testing the bioavailability and/or metabolism of compounds into active metabolites are also well-known. Ordinarily skilled artisans can routinely adapt such information to determine dosages of particular compounds suitable for human administration.

The compound(s) described herein, or compositions thereof, will generally be used in an amount effective to achieve the intended result, for example in an amount effective to treat or prevent the particular disease being treated. By therapeutic benefit is meant eradication, delaying onset or progression, or amelioration of the underlying disorder being treated and/or eradication, delaying onset or progression, or amelioration of one or more of the symptoms associated with the underlying disorder such that the patient reports an improvement in feeling or condition, notwithstanding that the patient may still be afflicted with the underlying disorder. Therapeutic benefit also generally includes halting or slowing the progression of the disease, regardless of whether improvement is realized.

### Examples

The preparation of the compounds of the disclosure (e.g., a compound of Formula (I), (I-A), (I-B), (I-C), (I-D), or Table 1) is illustrated by the following embodiments and examples, which are not to be construed as limiting the disclosure in scope or spirit to the specific procedures and compounds described in them.

### General Experimental

In general, compounds of the present invention are prepared by the method illustrated in the general reaction scheme described below, or by modifications thereof, using readily available starting materials, reagents, and conventional synthetic procedures. However, those skilled in the art will recognize that other methods may also be suitable. Also, in these reactions, it is possible to make use of variants that are in themselves known but are not mentioned here. Unless otherwise noted, all materials/reagents were obtained from commercial suppliers and used without further purification. Reactions were monitored by LC-MS and/or thin layer chromatography (TLC) on silica gel 60 F254 (0.2 mm) pre-coated aluminum foil or glass-backed and visualized using UV light. 1HNMR (400 MHz) spectra was recorded on Bruker spectrometers at room temperature (RT) with TMS or residual solvent peak as internal standard. The line positions or multiples are given in (δ) and coupling constants (J) are given as absolute values in Hertz (Hz). The multiplicities in 1HNMR spectra are abbreviated as follows: s (singlet), d (doublet), t (triplet), q (quartet), m (multiplet), br or broad (broadened). Preparative HPLC purification was performed on Shimadzu LC-6AD. All purification work was complete using Shim-pack PREP-DDS (H) KIT column. The mobile phases were water (with 0.1% HCO₂H) and acetonitrile. All reagents used were HPLC grade. The flow rate was 10 mL/min.

Preparative TLC was performed on Whatman LK6F Silica Gel 60A size 20×20 cm plates with a thickness of 1000 μm or equivalent. LC-MS was performed on Shimadzu LCMS-2020 equipped with LC-20AD or 30AD pumps, SPD-M20A PDA and Alltech 3300 ELSD. Mobile Phase A: water (0.1% formic acid); Mobile Phase B: acetonitrile (ACN); Duration: 5 minutes; Column: Sepax BR-C18 4.6*50 mm, 3 μm; Flow Rate: 1.0 mL/min; Oven Temperature: 40° C.

### Embodiment A. Synthesis of Compounds (1) and (5)

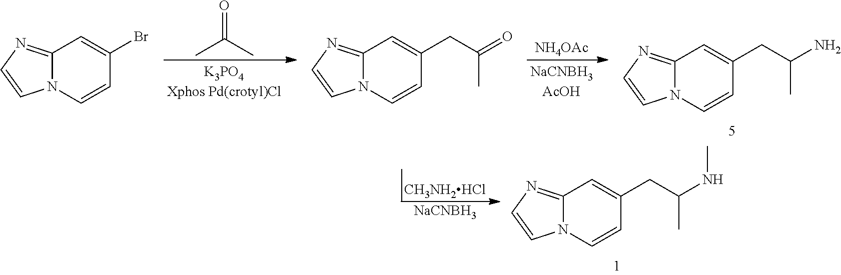

### Example—Compound 1

STEP 1: To a solution of 7-bromoimidazo[1,2-a]pyridine (200 mg, 1.02 mmol) and K₃PO₄ (646 mg, 3.05 mmol) in acetone (4 mL) was added XPhos Pd(crotyl)Cl (68 mg, 0.1 mmol) at RT under nitrogen. The mixture was stirred at 50° C. for 16 h. The reaction was diluted with 20 mL of dichloromethane, washed with saturated aqueous sodium chloride (2×20 mL), dried over anhydrous sodium sulfate, filtered and concentrated under reduced pressure. The residue was purified by reversed-phase flash chromatography (C18 silica gel column; mobile phase, acetonitrile in water (0.05% NH₄HCO₃), 10% to 100% gradient over 30 min) to afford the desired product (100 mg, 56%) as a white solid.

STEP 2: To a stirred solution of methanamine hydrochloride (106 mg, 1.57 mmol) in methanol (1 mL) was added NaBH₃CN (66 mg, 1.05 mmol) and the product from step 1 (100 mg, 0.52 mmol) at 0° C. and the resulting mixture was stirred RT for 16 h. The reaction was diluted with dichloromethane (20 mL). The organic layer was washed with saturated aqueous sodium chloride (2×20 mL), dried over anhydrous sodium sulfate. The resulting solution was concentrated under reduced pressure and purified by reverse-phase flash chromatography (C18 silica gel column; mobile phase, 0.05% aq. ammonium bicarbonate and acetonitrile, 20% acetonitrile up to 100% over 15 min, hold 100% for 5 min). 24 mg (22%) of Compound 1 was obtained as a white solid. MS m/z [M+H]⁺ (ESI): 190.05. ¹H NMR (300 MHz, CD₃OD) δ8.77 (d, J=6.9 Hz, 1H), 8.18 (d; J=1.8 Hz, 1H), 7.97 (d, J=2.1 Hz, 1H), 7.90 (s, 1H), 7.43-7.41 (m, 1H), 3.74-3.67 (m, 1H), 3.50-3.34 (m, 1H), 3.12-3.04 (m, 1H), 2.78 (s, 3H), 1.32 (d, J=6.6 Hz, 3H).

### Example—Compound 5

STEP 1 is performed as described above for Embodiment A. To a solution of the product from Step 1, Embodiment A (60 mg, 0.344 mmol) in MeOH (1.2 ml) was added NH₄OAc (80 mg, 1.03 mmol) and sodium cyanoborohydride (43 mg, 0.69 mmol) at 0° C. The mixture was stirred at room temperature for 16 h. The reaction was diluted with dichloromethane (20 mL), washed with saturated aqueous sodium chloride (2×20 mL), dried over anhydrous sodium sulfate, filtered and concentrated under reduced pressure. The crude product was purified by reverse phase flash chromatography (mobile phase, 0.05% aq. NH₄HCO₃, acetonitrile. 20% to 100% acetonitrile gradient over 15 min, hold 100% for 5 min) to afford Compound 5 (18 mg 30%) as a colorless semi-solid. MS m/z [M+H]⁺ (ESI): 176.05. ¹H NMR (300 MHz, CD₃OD) δ8.40-8.38 (m, 1H), 7.80-7.79 (m, 1H), 7.53 (d, J=1.2 Hz, 1H), 7.38 (s, 1H), 6.86-6.83 (m, 1H), 3.30-3.24 (m, 1H), 2.77 (d, J=6.6 Hz, 2H), 1.17 (d, J=6.3 Hz, 3H).

### Embodiment B. Synthesis of Compounds (2) and (6)

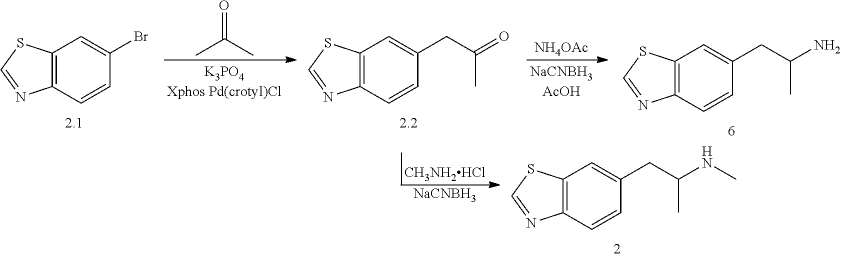

STEP 1: To a solution of 6-bromo-1,3-benzothiazole (200 mg, 0.93 mmol) and K₃PO₄ (397 mg, 1.87 mmol) in acetone (4 mL) was added XPhos Pd(crotyl)Cl (63 mg, 0.093 mmol) at RT under nitrogen. The resulting mixture was stirred for 16 h at 50° C. The reaction was diluted with dichloromethane (20 mL), washed with saturated aqueous sodium chloride (2×20 mL), dried over anhydrous sodium sulfate, filtered and concentrated under reduced pressure. The residue was purified by reversed-phase flash chromatography (mobile phase: acetonitrile in 0.05% aq. NH₄HCO₃, 10% to 100% acetonitrile gradient over 30 min) to afford the desired product (100 mg, 56%) as a yellow oil.

STEP 2: To a stirred solution of methanamine hydrochloride (106 mg, 1.57 mmol, 3 equiv) in methanol (1 mL) was added NaBH₃CN (66 mg, 1.05 mmol) and the product from Step 1 (100 mg, 0.52 mmol) at 0° C. The resulting mixture was stirred for 16 h at RT. The reaction was diluted with dichloromethane (20 mL). The organic layer was washed with saturated aqueous sodium chloride solution (2×20 mL) and dried over anhydrous sodium sulfate. The resulting solution was concentrated under reduced pressure. The crude product was purified by reverse phase HPLC (mobile phase: acetonitrile in 0.05% aq. NH₄HCO₃, 10% to 100% acetonitrile gradient over 30 min) to afford Compound 2 (37 mg, 72%) as a light yellow oil. MS m/z [M+H]⁺ (ESI): 207.00. ¹H NMR (400 MHz, DMSO-d₆) δ 9.35 (s, 1H), 8.05-8.03 (m, 2H), 7.44-7.42 (m, 1H), 3.26-3.17 (m, 2H), 2.78-2.72 (m, 1H), 2.50 (s, 3H), 1.06 (d, J=6.4 Hz, 3H).

### Example—Compound 6

Step 1 is performed as described above for Embodiment B. To a stirred solution of NH₄OAc (121 mg, 1.57 mmol) in methanol (1 mL) was added NaBH₃CN (66 mg, 1.05 mmol) and the product from Step 1, Embodiment B (100 mg, 0.52 mmol) at 0° C. The resulting mixture was stirred for 16 h at RT. The reaction was diluted with dichloromethane (20 mL), washed with saturated aqueous sodium chloride (2×20 mL), dried over anhydrous sodium sulfate, filtered and concentrated under reduced pressure. The crude product was purified by reverse phase HPLC (mobile Phase: 0.1% FA in water, acetonitrile; 3% to 20% acetonitrile gradient over 8 min then 20%) to afford Compound 6 (23 mg, 18%) as a white solid. MS m/z [M+H]⁺ (ESI): 193.00. ¹H NMR (400 MHz, DMSO-d₆) δ 9.35 (s, 1H), 8.04-8.02 (m, 2H), 7.42-7.40 (m, 1H), 3.40-3.32 (m, 1H), 3.02-2.98 (m, 1H), 2.82-2.75 (m, 1H), 1.08 (d, J=6.4 Hz, 3H).

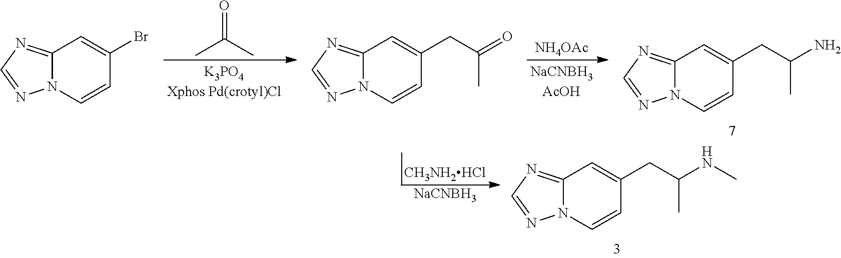

### Example—Compound 3

STEP 1: To a solution of 7-bromo-[1,2,4]triazolo[1,5-a]pyridine (200.0 mg, 1.01 mmol) and K₃PO₄ (429 mg, 2.02 mmol, 2) in acetone (4 mL) was added XPhos Pd(crotyl)Cl (68 mg, 0.101 mmol) at RT under nitrogen. The resulting mixture was stirred for 16 h at 50° C. The reaction was diluted with dichloromethane (20 mL), washed with saturated aqueous sodium chloride (2×20 mL), dried over anhydrous sodium sulfate, filtered and concentrated under reduced pressure. The residue was purified by reverse phase flash chromatography (mobile phase, acetonitrile in 0.05% aq. NH₄HCO₃, 10% to 100% acetonitrile gradient over 30 min) to afford the desired product (100 mg, 56%) as a yellow oil.

STEP 2: To a stirred solution of methanamine hydrochloride (116 mg, 1.71 mmol) in methanol (1 mL) was added NaBH₃CN (72 mg, 1.14 mmol) and the product from Step 1 (100 mg, 0.571 mmol) dropwise at 0° C. The resulting mixture was stirred for 16 h at RT. The reaction was diluted with dichloromethane (20 mL). The organic layer was washed with saturated aqueous sodium chloride (2×20 mL) and dried over anhydrous sodium sulfate. The resulting solution was concentrated under reduced pressure. The crude product was purified by reverse phase HPLC with the following conditions (mobile phase: 0.1% FA in water, acetonitrile; 5% to 30% acetonitrile gradient over 8 min then 30%) to afford Compound 3 (18 mg, 17%) as a colorless oil. MS m/z [M+H]⁺ (ESI): 190. 95. ¹H NMR (400 MHz, DMSO-d₆) δ 8.88 (d, J=6.8 Hz, 1H), 8.37 (s, 1H), 7.70 (s, 1H), 7.13-7.11 (m, 1H), 3.22-3.14 (m, 1H), 3.08-3.02 (m, 1H), 2.80-2.72 (m, 1H), 2.43 (s, 3H), 1.05 (d, J=6.4 Hz, 3H).

### Example—Compound 7

Step 1 is performed as described above for Embodiment C. To a stirred solution of NH₄OAc (150 mg, 1.94 mmol, 3 equiv) in methanol (1 mL) was added NaBH₃CN (82 mg, 1.30 mmol) and the product from Step 1, Embodiment C (100 mg, 0.57 mmol) at 0° C. The resulting mixture was stirred for 16 h at RT. The reaction was diluted with dichloromethane (20 mL), washed with saturated aqueous sodium chloride (2×20 mL), dried over anhydrous sodium sulfate, filtered and concentrated under reduced pressure. The crude product was purified by reverse phase HPLC (mobile phase: 0.1% FA in water, acetonitrile; 3% to 25% acetonitrile gradient over 8 min then 25%) to afford Compound 7 (16 mg, 11%) as a white solid. MS m/z [M+H]⁺ (ESI): 177.10. ¹H NMR (400 MHz, Methanol-d₄) δ 8.68 (d, J=7.2 Hz, 1H), 8.31 (s, 1H), 7.59 (s, 1H), 7.07-7.05 (m, 1H), 3.62-3.53 (m, 1H), 3.03-2.90 (m, 2H), 1.23 (d, J=6.8 Hz, 3H).

### Embodiment D. Synthesis of Compounds (4) and (8)

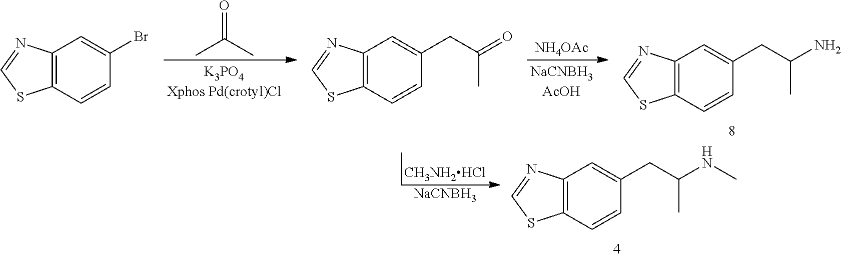

### Example—Compound 4

STEP 1: To a solution of 5-bromo-1,3-benzothiazole (200 mg, 0.93 PGP-65.03 mmol) and K₃PO₄ (397 mg, 1.87 mmol) in acetone (4 mL) was added XPhos Pd(crotyl)Cl (63 mg, 0.093 mmol) at RT under nitrogen. The resulting mixture was stirred for 16 h at 50° C. The reaction was diluted with dichloromethane (20 mL) washed with saturated aqueous sodium chloride (2×20 mL), dried over anhydrous sodium sulfate, filtered and concentrated under reduced pressure. The residue was purified by reverse phase flash chromatography (mobile phase, acetonitrile in 0.05% aq. NH₄HCO₃, 10% to 100% acetonitrile gradient over 30 min; detector) to afford the desired product (100 mg, 56%) as a yellow oil.

STEP 2: To a stirred solution of methanamine hydrochloride (106 mg, 1.57 mmol) in methanol (1 mL) was added NaBH₃CN (66 mg, 1.05 mmol) and the product from Step 1 (100 mg, 0.523 mmol) at 0° C. The resulting mixture was stirred for 16 h at RT. The reaction was diluted with dichloromethane (20 mL), washed with saturated aqueous sodium chloride (2×20 mL), dried over anhydrous sodium sulfate, filtered and concentrated under reduced pressure. The crude product was purified by HPLC (mobile phase: 0.1% FA in water:acetonitrile; 20% to 50% acetonitrile gradient over 8 min then 50%); to afford Compound 4 (21 mg, 16%) as a colorless oil. MS m/z [M+H]⁺ (ESI): 207.10. ¹H NMR (300 MHz, Methanol-d₄) δ 9.28 (s, 1H), 8.10-7.99 (m, 2H), 7.46-7.42 (m, 1H), 3.62-3.50 (m, 1H), 3.35-3.34 (m, 1H), 3.01-2.92 (m, 1H), 2.75 (s, 3H), 1.27 (d; J=6.6 Hz, 3H).

### Example—Compound 8

Step 1 is performed as described above for Embodiment D. To a stirred solution of NH₄OAc (121 mg, 1.57 mmol) in methanol (1 mL) was added NaBH₃CN (66 mg, 1.05 mmol) and the product from Step 1, Embodiment D (100 mg, 0.52 mmol) at 0° C. The resulting mixture was stirred for 16 h at RT. The reaction was diluted with dichloromethane (20 mL), washed with saturated aqueous sodium chloride (2×20 mL), dried over anhydrous sodium sulfate, filtered and concentrated under reduced pressure. The crude product was purified by reverse phase HPLC (mobile phase: water (0.1% FA), acetonitrile; 3% to 25% acetonitrile gradient over 8 min then 25%) to afford Compound 8 (28 mg, 22%) as an off-white solid. MS m/z [M+H]⁺ (ESI): 193.00. ¹H NMR (400 MHz, DMSO-d₆) δ 9.39 (s, 1H), 8.11 (d, J=8 Hz, 1H), 7.96 (s, 1H), 7.38-7.36 (m, 1H), 3.42-3.40 (m, 1H), 3.04-2.99 (m, 1H), 2.85-2.81 (m, 1H), 1.10 (d, J=6.4 Hz, 3H).

### Embodiment E. Synthesis of Compound 9

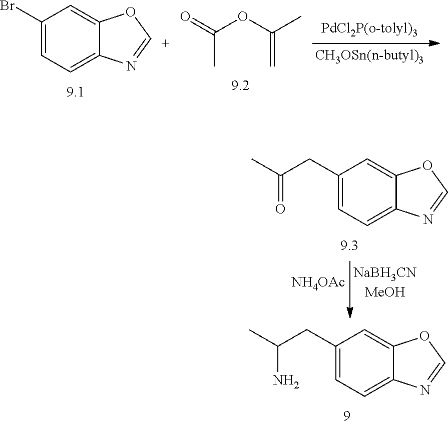

### Example—Compound 9

For step 1, to a solution of 6-bromo-1,3-benzoxazole, 9.1 (300 mg, 1.515 mmol, 1 equiv) in toluene (6 mL), prop-1-en-2-yl acetate, 9.2 (227.51 mg, 2.272 mmol, 1.5 equiv), tributyl(methoxy)stannane (729.68 mg, 2.272 mmol, 1.5 equiv) and tributyl(methoxy)stannane (729.68 mg, 2.272 mmol, 1.5 equiv) was added under nitrogen atmosphere. The mixture was stirred for 15 minutes. Palladium chloride (53.73 mg, 0.303 mmol, 0.2 equiv) was added. The mixture was stirred for overnight at 100° C. It was cooled to room temperature and concentrated under reduced pressure. Then the residue was dissolved in ethyl acetate and filtered through celite bed. The organic phase was washed with water, and saturated potassium fluoride solution, followed by brine solution. Combined organic layer was dried over anhydrous sodium sulfate, solvent was removed under vacuum and purified by silica gel column chromatography using petroleum ether/ethyl acetate (100%-60%, 15 minutes) as eluent to afford 9.3, 1-(benzo[d]oxazol-6-yl)propan-2-one (100 mg). MS m/z [M+H]⁺ (ESI): 176.05, which was used directly for the next step.

In this step, to a solution of NH₄OAc (49.50 mg, 0.642 mmol, 3 equiv), NaBH₃CN (26.90 mg, 0.428 mmol, 2 equiv) and acetic acid (catalyst) in methanol (0.5 mL) was added 9.3 (50 mg, 0.214 mmol, 1 equiv, 75%) in methanol (0.5 mL) at 0° C., dropwise. The mixture was stirred for 16 hours at room temperature. The residue was purified by Column: XBridge Prep OBD C18 Column, 30150 mm, 5 μm; Mobile Phase A: Water (10 mmol/L NH₄HCO₃), Mobile Phase B: acetonitrile; Flow rate: 60 mL/min mL/min; Gradient: 3% B to 20% B in 9 min; Wave Length: 220 nm nm; RT1(min): 6.68. This resulted desired 5.1 mg of product 9, 1-(benzo[d]oxazol-6-yl)propan-2-amine. MS m/z [M+H]⁺ (ESI):177.05. ¹H NMR (300 MHz, Methanol-d₄) b 8.45 (s, 1H), 7.72 (d, J=8.1 Hz, 1H), 7.58 (s, 1H), 7.35-7.25 (m, 1H), 3.53-3.38 (m, 1H), 3.06-2.80 (m, 2H), 1.22 (d, J=6.5 Hz, 3H).

### Embodiment F. Synthesis of Compounds (10) and (11)

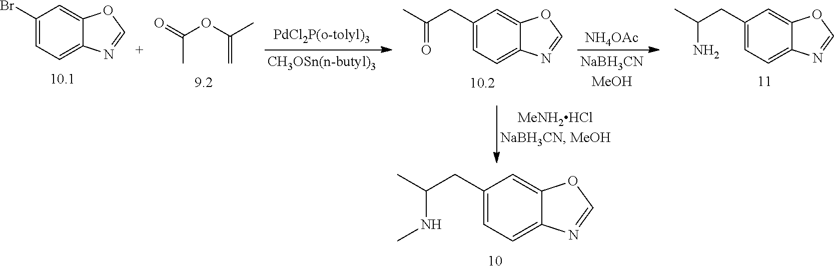

### Example—Compound 10

To a stirred solution of 6-bromo-2,1-benzoxazole, 10.1 (438.0 mg, 2.2 mmol, 1.0 equiv) in toluene was added prop-1-en-2-yl acetate, 9.2 (332.2 mg, 3.3 mmol, 1.5 equiv) and tributyl(methoxy)stannane (1065.3 mg, 3.3 mmol, 1.5 equiv) and PdCl₂ (78.4 mg, 0.4 mmol, 0.2 equiv) in portions at room temperature under argon atmosphere. The resulting mixture was stirred for additional 4 h at 100° C. After that time, the mixture was cooled to room temperature and concentrated under reduced pressure. The residue was dissolved in ethyl acetate and filtered through celite bed. The organic phase was washed with water, and saturated potassium fluoride solution, followed by brine solution. Combined organic layer was dried over anhydrous sodium sulfate, and solvent was removed under vacuum. The residue was purified by reversed-phase flash chromatography with the following conditions: column, C18 silica gel; mobile phase, MeCN in Water, 5% to 80% gradient in 30 min; detector, UV 254 nm. This resulted 140 mg of Compound 10.2, 1-(2,1-benzoxazol-6-yl) propan-2-one, which was used for the next step without further purification.

To a stirred solution of CH₃NH₂HCl (67.5 mg, 1.0 mmol, 2.5 equiv) in MeOH was added AcOH (0.07 mL, 1.2 mmol, 3.0 equiv) and NaBH₃CN (100.4 mg, 1.6 mmol, 4.0 equiv) in portions at 0° C. under argon atmosphere. To the above mixture was added 1-(2,1-benzoxazol-6-yl) propan-2-one, 10.2 (70.0 mg, 0.4 mmol, 1.0 equiv) dropwise over 1 min at 0° C. The resulting mixture was stirred for additional overnight at room temperature. The mixture was then concentrated under reduced pressure. The residue was purified by reversed-phase flash chromatography with the following conditions: column, C18 silica gel; mobile phase, MeCN in Water (10 mmol/L NH₄HCO₃), 5% to 80% gradient in 30 min; detector, UV 254 nm. The resulting mixture was concentrated under reduced pressure. The crude product (70.0 mg) was purified by Prep-H PLC with the following conditions (Column: YMC-Actus Triart C18 ExRS 30*150 mm, 5 μm; Mobile Phase A: Water (10 mmol/L NH₄HCO₃), Mobile Phase B: ACN; Flow rate: 60 mL/min mL/min; Gradient: 3% B to 15% B in 10 min; Wave Length: 220 nm/200 nm nm; RT1(min): 10.35) to afford 6.4 mg of Compound 10, [1-(2,1-benzoxazol-6-yl)propan-2-yl](methyl)amine. MS m/z [M+H]⁺ (ESI):191.10. ¹H NMR (400 MHz, DMSO-d6) δ 9.68 (s, 1H), 7.63 (d, J=9.0 Hz, 1H), 7.37 (s, 1H), 6.99-6.92 (m, 1H), 2.78 (dq, J=10.1, 5.8 Hz, 2H), 2.67 (s, 1H), 2.29 (s, 3H), 1.09 (s, 1H), 0.94 (d, J=5.8 Hz, 3H).

### Example—Compound 11

To a stirred solution of NH₄OAc (77.0 mg, 1.0 mmol, 2.5 equiv) in MeOH were added AcOH (0.07 mL, 1.2 mmol, 3.1 equiv) and NaBH₃CN (100.4 mg, 1.6 mmol, 4.0 equiv) in portions at 0° C. under argon atmosphere. To the above mixture was added compound 10.2, 1-(2,1-benzoxazol-6-yl) propan-2-one (70.0 mg, 0.4 mmol, 1.0 equiv) dropwise over 1 min at 0° C. The resulting mixture was stirred for additional overnight at room temperature. The resulting mixture was concentrated under reduced pressure. The residue was purified by reversed-phase flash chromatography with the following conditions: column, C18 silica gel; mobile phase, MeCN in Water (10 mmol/L NH₄HCO₃), 5% to 80% gradient in 30 min; detector, UV 254 nm. The resulting mixture was concentrated under reduced pressure. The crude product (70.0 mg) was purified by Prep-HPLC with the following conditions (Column: XBridge Prep Phenyl OBD Column 19250 mm, 5 m; Mobile Phase A: Water (10 mmol/L NH₄HCO₃), Mobile Phase B: ACN; Flow rate: 60 mL/min mL/min; Gradient: 0% B to 13% B in 10 min; Wave Length: 220 nm/200 nm nm; RT1(min): 8.22) to afford 17.2 mg of compound 11, 1-(2,1-benzoxazol-6-yl) propan-2-amine. MS m/z [M+H]⁺ (ESI): 177.20. ¹H NMR (400 MHz, DMSO-d6) δ 9.68 (d, J=1.1 Hz, 1H), 7.64 (d, J=8.9 Hz, 1H), 7.36 (s, 1H), 6.96 (dd, J=8.9, 1.3 Hz, 1H), 3.11 (q, J=6.5 Hz, 1H), 2.61 (d, J=6.6 Hz, 2H), 1.24 (s, 2H), 1.00 (d, J=6.3 Hz, 3H).

### Embodiment G. Synthesis of Compounds (12) and (13)

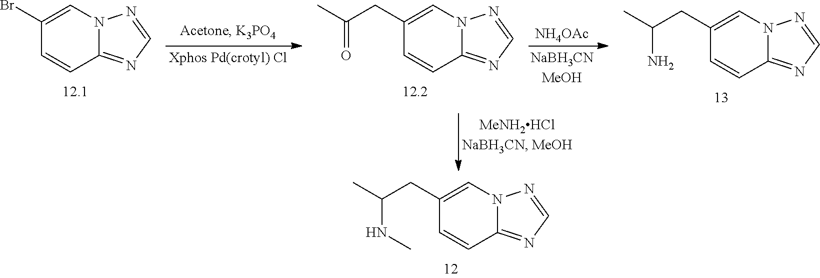

### Example—Compound 12

To a solution of 6-bromo-[1,2,4]triazolo[1,5-a]pyridine, 12.1 (400 mg, 2.020 mmol, 1 equiv) in acetone (8 mL) was added (phosphoperoxy)potassium; dipotassium (1.29 g, 6.060 mmol, 3 equiv) and Xphos Pd(crotyl)Cl (136.15 mg, 0.202 mmol, 0.1 equiv) under nitrogen atmosphere. The mixture was stirred for 16 hours at 50° C. Subsequently, the mixture was cooled to room temperature and filtered, washing the filter cake with dichloromethane. The filtrate was concentrated under reduced pressure. The residue was purified by silica gel column chromatography, eluted with petroleum ether/ethyl acetate (100%-0, 15 minutes) to afforded 200 mg of intermediate 12.2. MS m/z [M+H]⁺ (ESI):176.15, which was used directly next step without further characterization.

To a mixture of methylamine hydrochloride (115.62 mg, 1.713 mmol, 3 equiv) and NaBH₃CN (71.74 mg, 1.142 mmol, 2 equiv) in methanol (1 mL) was added a solution of 12.2 (100 mg, 0.571 mmol, 1 equiv) in methanol (1 mL) by dropwise at 0° C. Then, the mixture was stirred for 4 hours at room temperature. The residue was purified twice by Column: Xselect CSH C18 OBD Column, 30*150 mm, 5 μm; Mobile Phase A: Water (0.1% FA), Mobile Phase B: ACN; Flow rate: 60 mL/min; Gradient: 3% B to 25% B in 10 min; Wave Length: 254 nm; RT1(min): 9. This resulted in 34.7 mg of compound 12; methyl(1-{[1,2,4]triazolo[1,5-a]pyridin-6-yl}propan-2-yl)amine. MS m/z [M+H]⁺ (ESI):191.00. ¹H NMR (300 MHz, Methanol-d₄) δ8.79 (s, 1H), 8.42 (s, 1H), 7.80 (d, J=9.3 Hz, 1H), 7.72-7.61 (m, 1H), 3.70-3.47 (m, 1H), 3.29-3.18 (m, 1H), 3.01-2.87 (m, 1H), 2.76 (s, 3H), 1.31 (d, J=6.6 Hz, 3H).

### Example—Compound 13

To a mixture of NH₄OAc (132.00 mg, 1.713 mmol, 3 equiv) and NaBH₃CN (71.74 mg, 1.142 mmol, 2 equiv) in methanol (1 mL) was added a solution of 12.2 (100 mg, 0.571 mmol, 1 equiv) in methanol (1 mL) by dropwise at 0° C. Then, the mixture was stirred for 4 hours at room temperature. The residue was purified by Column: XBridge Prep OBD C18 Column, 30*150 mm, 5 μm; Mobile Phase A: Water (10 mmol/L NH₄HCO₃₊0.05% NH₃·H₂O), Mobile Phase B: ACN; Flow rate: 60 mL/min; Gradient: 3% B to 20% B in 9 min; Wave Length: 220 nm; RT1(min): 5.57. This resulted 33.2 mg of compound 13, methyl(1-{[1,2,4]triazolo[1,5-a]pyridin-6-yl}propan-2-yl)amine. MS m/z [M+H]⁺ (ESI):177.25. ¹H NMR (300 MHz, Methanol-d₄) δ 8.67 (s, 1H), 8.37 (s, 1H), 7.74 (d, J=9.3 Hz, 1H), 7.67-7.56 (m, 1H), 3.29-3.21 (m, 1H), 2.88-2.74 (m, 2H), 1.18 (d, J=6.4 Hz, 3H).

### Embodiment H. Synthesis of Compounds (14), (15), (16) and (17)

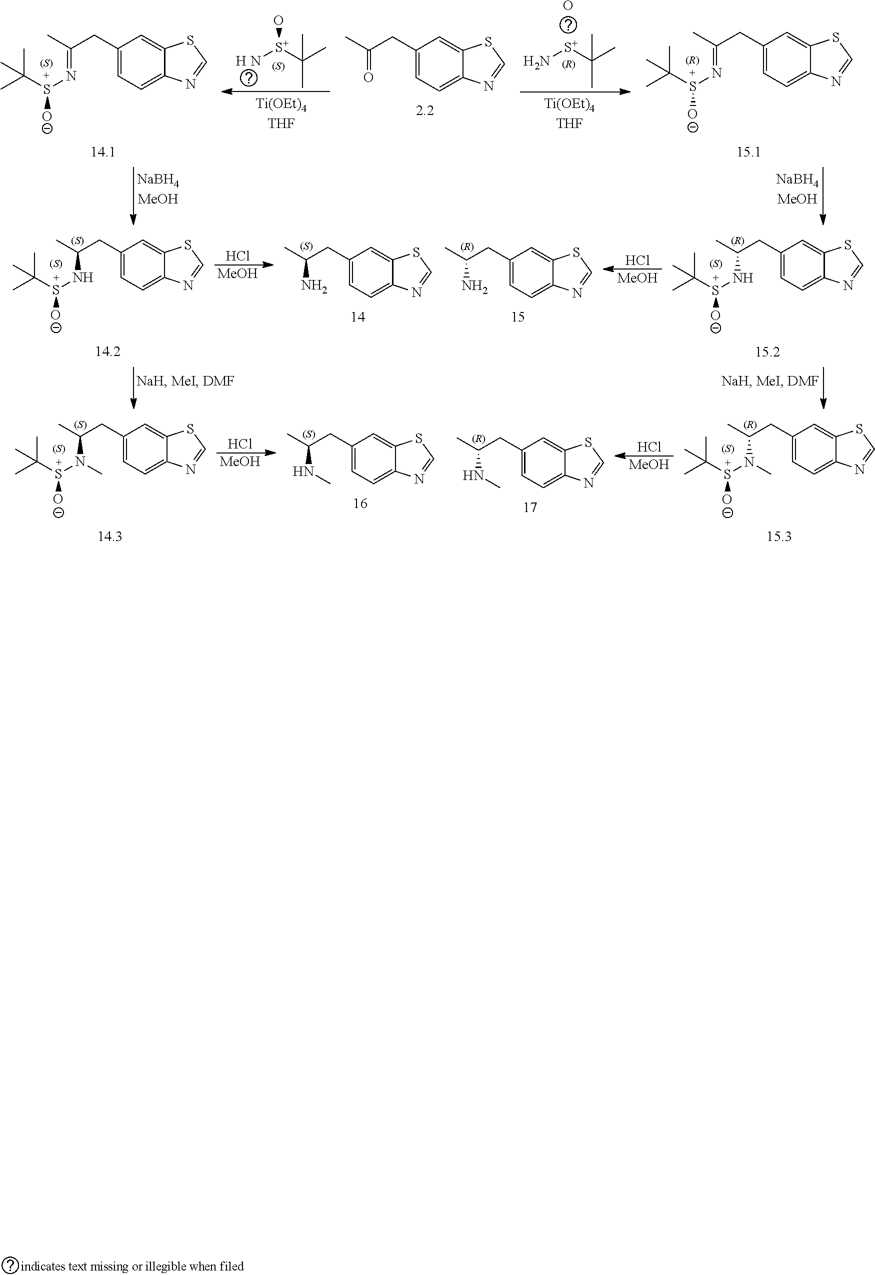

### Example—Compound 14

To a solution of compound 2.2 (370 mg, 1.935 mmol, 1 equiv) in tetrahydrofuran (3.7 mL) was added (S)-2-methylpropane-2-sulfinamide (257.93 mg, 2.129 mmol, 1.1 equiv) and tetraethoxytitanium (706.10 mg, 3.096 mmol, 1.6 equiv) under nitrogen atmosphere. The mixture was stirred for 2 hours at 66° C. Desired product 14.1, is detectable by LCMS. MS m/z [M+H]⁺ (ESI):295.05. This crude material was used for the next reaction without further purification.

To the solution of 14.1 in tetrahydrofuran was added NaBH₄ (146.22 mg, 3.865 mmol, 2.00 equiv) at −30° C. The mixture was stirred for 30 minutes at −30° C. Acetone was added slowly followed by methanol and the reaction allowed to warm to room temperature. To the reaction mixture was added water and stirred for 1 hours before filtering the titanium salts. The cake was washed with 2-MeTHF and the filtrate concentrated to remove tetrahydrofuran and methanol. The residue was purified by reversed-phase flash chromatography with the following conditions: column, C18 silica gel; mobile phase, acetonitrile in water (0.05% NH₄HCO₃), 10% to 50% gradient in 10 min; detector, UV 254 nm. This resulted in 200 mg of intermediate 14.2. MS m/z [M+H]⁺ (ESI):297.00. This crude material was used for the next reaction without further purification.

To HCl in MeOH (1.6 mol/L, 1.6 mL) was added 14.2 (80 mg, 0.270 mmol, 1 equiv). The solution was stirred for one hour at room temperature. The solvent was removed under reduced pressure and the residue was washed with diethyl ether. This resulted 35.7 mg of 14, (S)-1-(benzo[d]thiazol-6-yl)propan-2-amine. MS m/z [M+H]⁺ (ESI):193.05. ¹H NMR (300 MHz, Methanol-d₄) δ 10.02 (s, 1H), 8.28-8.01 (m, 2H), 7.80-7.63 (m, 1H), 3.83-3.60 (m, 1H), 3.29-3.21 (m, 1H), 3.14-3.00 (m, 1H), 1.32 (d, J=6.6 Hz, 3H). [α]_{D}^{24.8}=+33.3.

### Example—Compound 16

To a solution of 14.2 (100 mg, 0.337 mmol, 1 equiv) in dimethylformamide (2 mL) was added sodium hydride (20.24 mg, 0.506 mmol, 1.5 equiv, 60% in oil) at 0° C. The mixture was stirred for 15 minutes at room temperature. Methyl iodide (71.82 mg, 0.506 mmol, 1.5 equiv) was added at 0° C. and the mixture was stirred for one hour at room temperature. The reaction was quenched with sat. NH₄Cl and extracted with DCM. The organic phase was washed with water and brine, dried over anhydrous Na₂SO₄, and concentrated under reduced pressure. The residue was purified by reversed-phase flash chromatography with the following conditions: column, C18 silica gel; mobile phase, ACN in water (0.05% NH₄HCO₃), 20% to 100% gradient in 20 min; detector, UV 254 nm. This resulted 100 mg of compound 14.3. MS m/z [M+H]⁺ (ESI):311.10. This crude material was used for the next reaction without further purification.

To HCl in MeOH (1.6 mol/L, 1.6 mL) was added 14.3 (80 mg, 0.258 mmol, 1 equiv). The solution was stirred for one hour at room temperature. The solvent was removed under reduced pressure and the residue was purified by trituration with diethyl ether. The residue was purified by Column: Atlantis Prep T3 OBD Column, 19*250 mm 10 μm; Mobile Phase A: Water (0.1% HCl), Mobile Phase B: ACN; Flow rate: 20 mL/min mL/min; Gradient: 3% B to 25% B in 9 min; Wave Length: 254 nm nm; RT1(min): 7. This resulted 35.9 mg of 16, (S)-1-(benzo[d]thiazol-6-yl)-N-methylpropan-2-amine. MS m/z [M+H]⁺ (ESI):207.05. ¹H NMR (300 MHz, Methanol-d₄) δ 10.07 (s, 1H), 8.25 (s, 1H), 8.16 (d, J=8.4 Hz, 1H), 7.81-7.67 (m, 1H), 3.72-3.52 (m, 1H), 3.51-3.35 (m, 1H), 3.12-2.94 (m, 1H), 2.77 (s, 3H), 1.28 (d, J=6.6 Hz, 3H).

### Example—Compound 15

To a solution of 2.2 (400 mg, 2.092 mmol, 1 equiv) in tetrahydrofuran (THF, 4 mL) was added (R)-2-methylpropane-2-sulfinamide (278.84 mg, 2.301 mmol, 1.1 equiv) and tetraethoxytitanium (763.35 mg, 3.347 mmol, 1.6 equiv) under nitrogen atmosphere. The mixture was stirred for 2 hours at 66° C. The crude material 15.1 showed desired mass (MS m/z [M+H]⁺ (ESI):295.05) and was used for the next step without further purification.

To the solution of 15.1 in tetrahydrofuran (from previous step) was added NaBH₄ (86.92 mg, 2.298 mmol, 1.1 equiv) at −30° C. The mixture was stirred for 30 minutes at −30° C. Acetone was added slowly followed by methanol and the reaction allowed to warm to room temperature. To the reaction mixture was added water and stirred for 1 h before filtering the titanium salts. The cake was washed with 2-MeTHF and the filtrate concentrated to remove tetrahydrofuran and methanol. The residue was purified by reversed-phase flash chromatography with the following conditions: column, C18 silica gel; mobile phase, acetonitrile in water (0.05% NH₄HCO₃), 10% to 50% gradient in 10 min; detector, UV 254 nm. This resulted 180 mg of compound 15.2. MS m/z [M+H]⁺ (ESI):297.00. This was used for the next step without further purification.

To HCl in MeOH(1.6 mol/L, 1.6 mL) was added compound 15.2 (80 mg, 0.270 mmol, 1 equiv). The solution was stirred for one hour at room temperature. The solvent was removed under reduced pressure and the residue was washed with diethyl ether. This resulted 37.1 mg of 15, (R)-1-(benzo[d]thiazol-6-yl)propan-2-amine. MS m/z [M+H]+(ESI):193.05. ¹H NMR (300 MHz, Methanol-d₄) δ 9.66 (s, 1H), 8.14-8.07 (m, 2H), 7.63-7.56 (m, 1H), 3.73-3.57 (m, 1H), 3.25-3.14 (m, 1H), 3.09-2.95 (m, 1H), 1.31 (d, J=6.6 Hz, 3H). [α]_{D}^{23.6}=−12.3°.

### Example—Compound 17

To a solution of 15.2 (100 mg, 0.337 mmol, 1 equiv) in dimethylformamide (DMF, 2 mL) was added sodium hydride (20.24 mg, 0.506 mmol, 1.5 equiv, 60% in oil) at 0° C. The mixture was stirred for 15 minutes at room temperature. methyl iodide (71.82 mg, 0.506 mmol, 1.5 equiv) was added at 0° C. and the mixture was stirred for one hour at room temperature. The reaction was quenched with sat. NH₄Cl and extracted with DCM. The organic phase was washed with water and brine, dried over anhydrous Na₂SO₄, and concentrated under reduced pressure. The residue was purified by reversed-phase flash chromatography with the following conditions: column, C18 silica gel; mobile phase, ACN in water (0.05% NH₄HCO₃), 20% to 100% gradient in 20 min; detector, UV 254 nm. This resulted 100 mg of compound 15.3. MS m/z [M+H]⁺ (ESI):311.10. This crude material was used for the next reaction without further purification.

To HCl in MeOH (1.6 mol/L, 1.6 mL) was added compound 15.3 (80 mg, 0.258 mmol, 1 equiv). The solution was stirred for one hour at room temperature. The solvent was removed under reduced pressure and the residue was washed with diethyl ether. This resulted 38.7 mg of 17, (R)-1-(benzo[d]thiazol-6-yl)-N-methylpropan-2-amine. MS m/z [M+H]⁺ (ESI):207.05. ¹H NMR (300 MHz, Methanol-d₄) δ 9.86 (s, 1H), 8.23-8.08 (m, 2H), 7.71-7.63 (m, 1H), 3.72-3.53 (m, 1H), 3.47-3.33 (m, 1H), 3.08-2.96 (m, 1H), 2.77 (s, 3H), 1.28 (d, J=6.6 Hz, 3H).

### Examples—Biological Activity

### Assay 1—Human Serotonin Transporter (SERT) Uptake Inhibition Assay.

Compound activity is assessed using an in vitro assay. Briefly, HEK cells expressing recombinant serotonin transporter (SERT) are plated at 50,000 cells/well on a 96 well plate pre-coated with Matrigel one day prior to the experiment. Culture medium (DMEM/FBS) is removed and 30 μl of assay buffer (Tris-HCl 50 mM, EDTA 4 mM, BDA 0.1%) with the desired concentration of test compound is added. The plate is incubated at 37° C. for 15 minutes. Assay buffer (30 μl) containing the same concentration of compound diluted in [³H]-Serotonin uptake buffer (final concentration 10 nM) is added to each well and the plate is incubated at 37° C. for 5 minutes. The reaction mixture is removed, and the cells are washed with 100 μl ice-cold assay buffer twice. Lysis buffer (50 μl) is added to the cells followed by a 5 minute incubation with gentle shaking at room temperature. The lysate is transferred to a 96 well isoplate. Optiphase supermix (100 μl) is added to each well with complete mixing. Radioactivity is counted with Microbeta Counter and reported as counts per minute (CPM). Fluoxetine is used as a control to measure non-specific uptake and for data normalization. Percent inhibition of [³H]-Serotonin uptake calculations: 100×(1−(CPM_{test sample}−CPM_{non_specific uptake})/(CPM_{MAX}−CPM_{MIN})) are summarized in Table 2. Subsequent experiments using the same assay were performed with select compounds with a greater range of concentrations (10 half-log dilutions from a top concentration of 100 μM), allowing for the determination of IC50s. IC50s were determined using the log(inhibitor) vs. normalized response—Variable slope model in GraphPad Prism 10.1.0 and are summarized in Table 3.

TABLE 2
SERT Uptake Inhibition (N = 1-4, results from individual
experiments are separated by commas)

| | | SERT | SERT | SERT |
|---|---|---|---|---|
| | | UPTAKE | UPTAKE | UPTAKE |
| Cmpd | | INHIBITION | INHIBITION | INHIBITION |
| ID | Structure | (%) @100 uM | (%) @10 uM | (%) @1 uM |
| 1 | 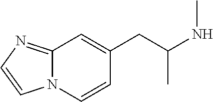 | 91.6 | | 3.6 |
| 2 | 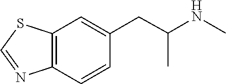 | 88.75, 93.94, 91.08 | 67.9 | 43.44, −5.16 |
| 3 | 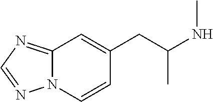 | 78.37, 69.13 | | −1.21, −1.77 |
| 4 | 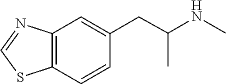 | 91.55, 87.43 | 61.8 | 20.2 |
| 5 | 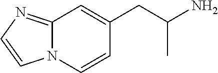 | 90.2 | | −0.48 |
| 6 | 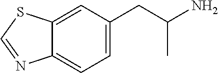 | 94.61, 96.89 | | 51.1, 4.94 |
| 7 | 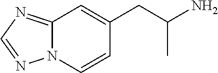 | 84.23, 59.35 | | 11.89, −1.06 |
| 8 | 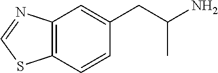 | 82.37, 90.93 | 68.7 | 24.0 |
| 9 | 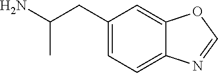 | 87.9 | −2.39 | |
| 10 | 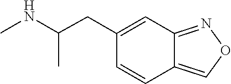 | 96.8 | 48.3 | |
| 11 | 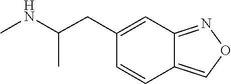 | 96.6 | 45.5 | |
| 12 | 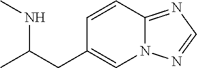 | 83.15 | 0.25 | |
| 13 | 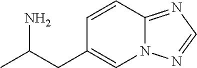 | 73.14 | 1.34 | |
| 14 | 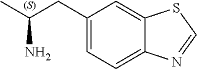 | 98.9 | 46.6 | |
| 15 | 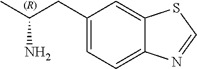 | 97.5 | 38.0 | |
| 16 | 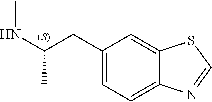 | 99.1 | 57.7 | |
| 17 | 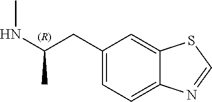 | 95.7 | 32.5 | |
| 18 MDMA | 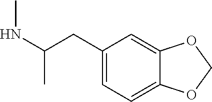 | 85.1, 88.7, 98.0, 86.2 | 53.3, 70.1 | 25.0, −1.67 |

### Assay 2—Human Dopamine Transporter (DAT) Uptake Inhibition Assay.

Compound activity is assessed using an in vitro assay. Briefly, HEK cells expressing recombinant dopamine transporter (DAT) are plated at 50,000 cells/well on a 96 well plate pre-coated with Matrigel one day prior to the experiment. Culture medium (DMEM/FBS) is removed and 30 μl of assay buffer (Tris-HCl 50 mM, EDTA 4 mM, BDA 0.1%) with the desired concentration of test compound is added. The plate is incubated at 37° C. for 15 minutes. Assay buffer (30 μl) containing the same concentration of compound diluted in [³H]-Dihydroxyphenylethylamine uptake buffer (final concentration 20 nM) is added to each well and the plate is incubated at 37° C.; for 5 minutes. The reaction mixture is removed, and the cells are washed with 100 μl ice-cold assay buffer twice. Lysis buffer (50 μl) is added to the cells followed by a 5 minute incubation with gentle shaking at room temperature. The lysate is transferred to a 96 well isoplate. Optiphase supermix (100 μl) is added to each well with complete mixing. Radioactivity is counted with Microbeta Counter and reported as CPM. Nomifensine is used as a control to measure non-specific uptake and for data normalization. Percent inhibition of [³H]-Dihydroxyphenylethylamine uptake calculations: 100×(1−(CPM_{test sample}−CPM_{non-specific uptake})/(CPM_{MAX}−CPM_{MIN})). Test compounds were screened at 10 concentrations at half-log dilutions from a top concentration of 100 μM. IC50s were determined using the log(inhibitor) vs. normalized response—Variable slope model in GraphPad Prism 10.1.0 and are summarized in Table 3.

### Assay 3—Human Norepinephrine Transporter (NET) Uptake Inhibition Assay.

Compound activity is assessed using an in vitro assay. Briefly, HEK cells expressing recombinant Norepinephrine transporter (NET) are plated at 50,000 cells/well on a 96 well plate pre-coated with Matrigel one day prior to the experiment. Culture medium (DMEM/FBS) is removed and 30 μl of assay buffer (Tris-HCl 50 mM, EDTA 4 mM, BDA 0.1%) with the desired concentration of test compound is added. The plate is incubated at 37° C. for 15 minutes. Assay buffer (30 μl) containing the same concentration of compound diluted in [³H]-Norepinephrine uptake buffer (final concentration 20 nM) is added to each well and the plate is incubated at 37° C. for 5 minutes. The reaction mixture is removed, and the cells are washed with 100 μl ice-cold assay buffer twice. Lysis buffer (50 μl) is added to the cells followed by a 5 minute incubation with gentle shaking at room temperature. The lysate is transferred to a 96 well isoplate. Optiphase supermix (100 μl) is added to each well with complete mixing. Radioactivity is counted with Microbeta Counter and reported as CPM. Desiparmine is used as a control to measure non-specific uptake and for data normalization. Percent inhibition of [³H]-Norepinephrine uptake calculations: 100×(1−CPM_{test sample}−CPM_{non_specific uptake})/(CPM_{MAX}−CPM_{MIN})). Test compounds were screened at 10 concentrations at half-log dilutions from a top concentration of 100 μM. IC50s were determined using the log(inhibitor) vs. normalized response—Variable slope model in GraphPad Prism 10.1.0 and are summarized in Table 3.

TABLE 3
Summary of SERT, DAT and NET Uptake Inhibition IC50s (N = 1-6,
results from individual experiments are separated by commas)

| | | SERT | DAT | NET |
|---|---|---|---|---|
| | | Uptake | Uptake | Uptake |
| Cmpd | | Inhibition | Inhibition | Inhibition |
| ID | Structure | IC50 (uM) | IC50 (uM) | IC50 (uM) |
| 1 | 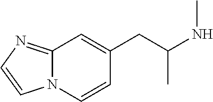 | | | |
| 2 | 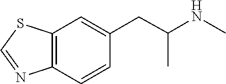 | 10.3 | 21.8 | 4.7 |
| 3 | 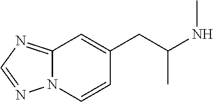 | | | |
| 4 | 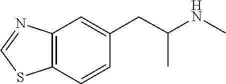 | 11.3 | 35.8 | 8.3 |
| 5 | 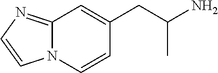 | | | |
| 6 | 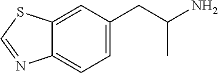 | 7.5 | >100 | 1.1 |
| 7 | 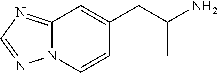 | | | |
| 8 | 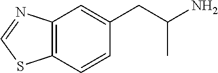 | 11.5 | 20.7 | >100 |
| 9 | 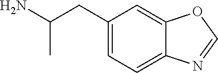 | | | |
| 10 | 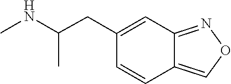 | | | |
| 11 | 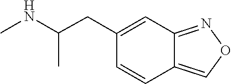 | | | |
| 12 | 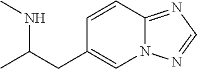 | | | |
| 13 | 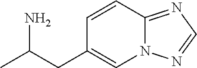 | | | |
| 14 | 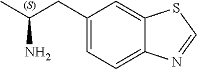 | 6.6 | >100 | 5.8 |
| 15 | 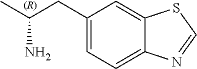 | | | |
| 16 | 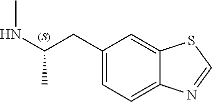 | 5.1 | >100 | 4.2 |
| 17 | 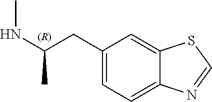 | | | |
| 18- MDMA (Cntrl) | 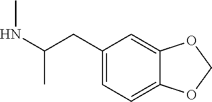 | 5.25, 5.08, 3.19, 1.48, 2.07, 2.21 | 3.50, 5.48, 5.29, 7.48, 10.28, 13.54 | 1.40, 2.37, 0.93, 1.34, 2.27, 2.89 |

It is understood that the examples and embodiments described herein are for illustrative purposes only and that various modifications or changes in light thereof will be suggested to persons skilled in the art and are to be incorporated within the spirit and purview of this application and scope of the appended claims. All publications, patents, and patent applications cited herein are hereby incorporated herein by reference for all purposes.

We claim:

1. A compound of Formula (I): 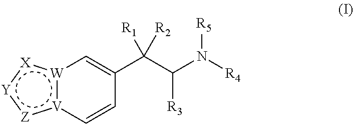
or a pharmaceutically acceptable salt thereof,
wherein,
V and W independently are C or N;
X, Y, and Z independently are O, S, CR₆, N, or NR₇;
wherein R₆ is hydrogen, deuterium, halogen, alkyl, fluoroalkyl, O-alkyl, O-fluoroalkyl, aryl, or benzyl;
wherein R₇ is hydrogen, deuterium, alkyl, or fluoroalkyl;
R₁ and R₂ independently are hydrogen, deuterium, halogen, alkyl, fluoroalkyl, O-alkyl, or O-fluoroalkyl; or
R₁ and R₂ together are carbonyl; or
R₁ and R₂ together with the carbon atom to which they are attached form a cycloalkyl;
R₃ is hydrogen, deuterium, halogen, alkyl, fluoroalkyl, O-alkyl, or O-fluoroalkyl; and
R₄ and R₅ independently are hydrogen, deuterium, alkyl, or fluoroalkyl; or
one of R₄ or R₅ is 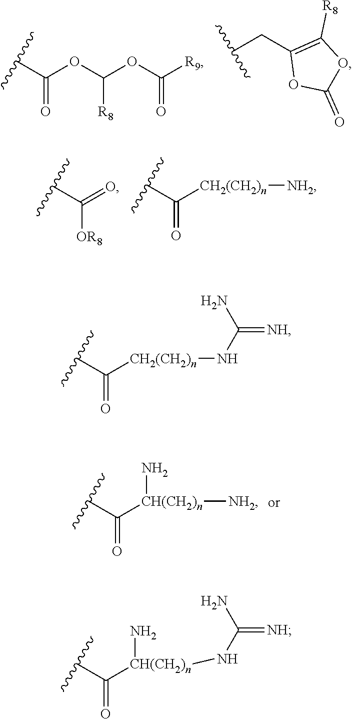
wherein R₈ and R₉ independently are alkyl; and
wherein n is an integer from 2-5.

2. The compound of claim 1, wherein
X, Y, and Z independently are CR₆ or NR₇;
wherein R₆ is C₁-C₆ alkyl, C₁-C₆ fluoroalkyl, O—(C₁-C₆ alkyl), or O—(C₁-C₆ fluoroalkyl);
wherein R₇ is C₁-C₆ alkyl or C₁-C₆ fluoroalkyl;
R₁ and R₂ independently are C₁-C₆ alkyl, C₁-C₆ fluoroalkyl, O—(C₁-C₆ alkyl), or O—(C₁-C₆ fluoroalkyl); or
R₁ and R₂ together with the carbon atom to which they are attached form a C₃-C₇ cycloalkyl;
R₃ is C₁-C₆ alkyl, C₁-C₆ fluoroalkyl, O—(C₁-C₆ alkyl), or O—(C₁-C₆ fluoroalkyl); and
R₄ and R₅ independently are C₁-C₆ alkyl or C₁-C₆ fluoroalkyl; or
one of R₄ or R₅ is 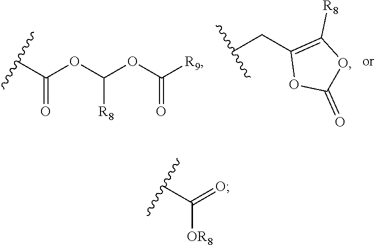
wherein R₈ and R₉ independently are C₁-C₆ alkyl.

3. The compound of any of claims 1-2, wherein R₃ is C₁-C₆ alkyl.

4. The compound of claim 3, wherein R₃ is methyl.

5. The compound of any of claims 1-4, wherein R₄ or R₅ is C₁-C₆ alkyl.

6. The compound of claim 5, wherein R₄ or R₅ is methyl.

7. The compound of any of claims 1-4, wherein R₄ and R₅ are hydrogen.

8. The compound of any of claims 1-7, wherein at least two of V, W, X, Y, and Z are selected from N and S.

9. The compound of any of claims 1-8, wherein at least three of V, W, X, Y, and Z are selected from N and S.

10. The compound of any of claims 1-9, wherein V and X are N.

11. The compound of any of claims 1-9, wherein X is S and Z is N.

12. The compound of any of claims 1-9, wherein V, X, and Z are N.

13. The compound of any of claims 1-9, wherein X is N and Z is S.

14. The compound of any of claims 1-9, wherein V is C.

15. The compound of any of claims 1-9, wherein V is N.

16. The compound of any of claims 1-9, wherein W is C.

17. The compound of any of claims 1-9, wherein X is N.

18. The compound of any of claims 1-9, wherein X is S.

19. The compound of any of claims 1-9, wherein Y is CH.

20. The compound of any of claims 1-9, wherein Z is CH.

21. The compound of any of claims 1-9, wherein Z is N.

22. The compound of any of claims 1-9, wherein Z is S.

23. The compound of any of claims 1-22, wherein R₁ and R₂ are hydrogen.

24. A compound of Formula (I-A): 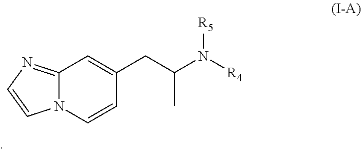
or a pharmaceutically acceptable salt thereof,
wherein,
R₄ and R₅ independently are hydrogen, deuterium, C₁-C₆ alkyl or C₁-C₆ fluoroalkyl; or
one of R₄ or R₅ is 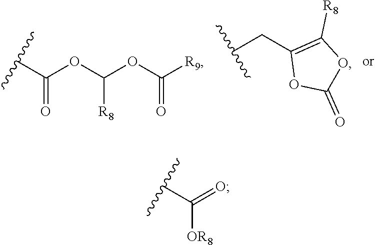
wherein R₈ and R₉ independently are C₁-C₆ alkyl.

25. A compound of Formula (I-B): 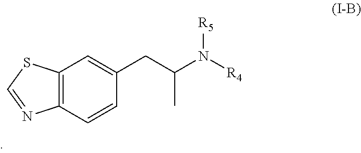
or a pharmaceutically acceptable salt thereof,
wherein,
R₄ and R₅ independently are hydrogen, deuterium, C₁-C₆ alkyl or C₁-C₆ fluoroalkyl; or
one of R₄ or R₅ is 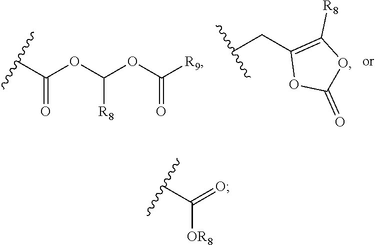
wherein R₈ and R₉ independently are C₁-C₆ alkyl.

26. A compound of Formula (I-C): 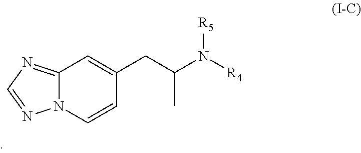
or a pharmaceutically acceptable salt thereof,
wherein,
R₄ and R₅ independently are hydrogen, deuterium, C₁-C₆ alkyl or C₁-C₆ fluoroalkyl; or
one of R₄ or R₅ is 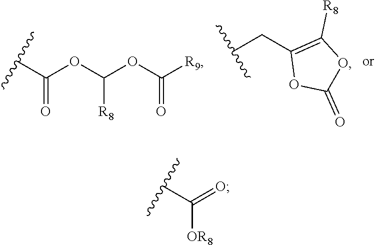
wherein R₈ and R₉ independently are C₁-C₆ alkyl.

27. A compound of Formula (I-D): 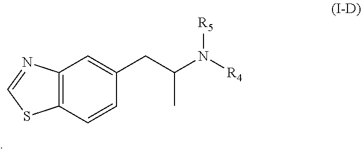
or a pharmaceutically acceptable salt thereof,
wherein,
R₄ and R₅ independently are hydrogen, deuterium, C₁-C₆ alkyl or C₁-C₆ fluoroalkyl; or
one of R₄ or R₅ is 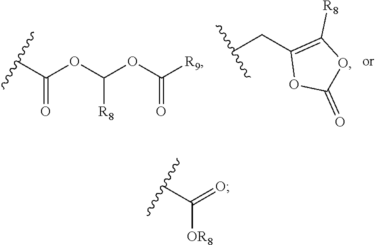
wherein R₈ and R₉ independently are C₁-C₆ alkyl.

28. The compound of any of claims 1-23, wherein the compound is not:

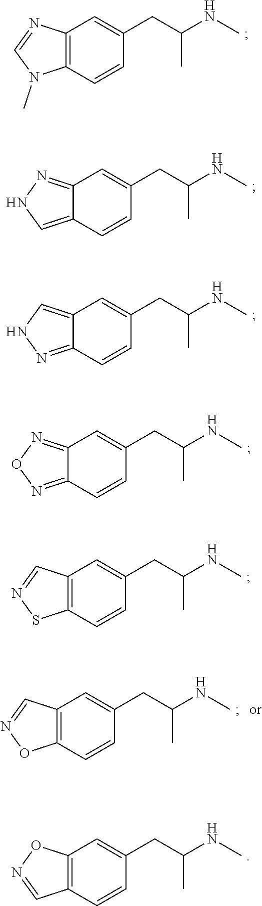

29. A compound, or a pharmaceutically acceptable salt thereof, selected from:

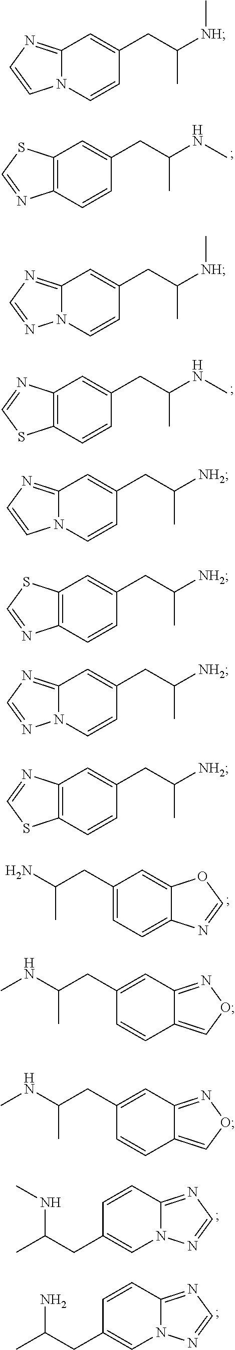

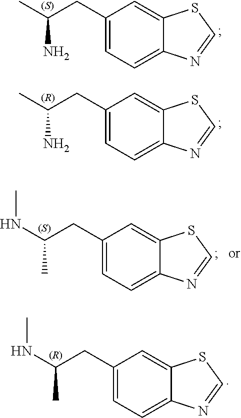

30. A composition comprising the compound of any of claims 1-29 and a pharmaceutically acceptable carrier.

31. The compound of any of claims 1-29 for use as a medicament.

32. A method of treating a neurological disorder, comprising administering to a subject in need thereof an effective amount of the compound of any of claims 1-29 or the composition of claim 30.

33. The method of claim 32, wherein the neurological disorder is post-traumatic stress disorder.

34. The method of claim 32, wherein the neurological disorder is an eating disorder.

35. The method of claim 34, wherein the eating disorder is anorexia nervosa or bulimia nervosa.

36. The method of claim 32, wherein the neurological disorder is borderline personality disorder, a Cluster C personality disorder, or obsessive-compulsive disorder (OCD).

37. The compound of any of claims 1-29 or the composition of claim 30 for use in the treatment of a neurological disorder.

38. The compound of any of claims 1-29 or the composition of claim 30 for use in the treatment of post-traumatic stress disorder.

39. The compound of any of claims 1-29 or the composition of claim 30 for use in the treatment of an eating disorder.

40. The compound of any of claims 1-29 or the composition of claim 30 for use in the treatment of anorexia nervosa or bulimia nervosa.

41. The compound of any of claims 1-29 or the composition of claim 30 for use in the treatment of borderline personality disorder, a Cluster C personality disorder, or obsessive-compulsive disorder (OCD).
